(19) United States
(12) Patent Application Publication
(10) Pub. No.: US 2025/0069722 A1
(43) Pub. Date: Feb. 27, 2025

(54) IDENTIFICATION OF ORGAN DONORS FOR TRANSPLANTATION AMONG POTENTIAL DONORS

(71) Applicant: ORTHO BIOMED INC., Toronto (CA)

(72) Inventors: Nick SAJADI, Toronto (CA); Mohammad Ali SHAFIEE NYESTANAK, Richmond Hill (CA); Ebrahim POURJAFARI, Toronto (CA); Seyed Hamid Reza MIRKHANI, Thornhill (CA); Seyed Mohammad ALAVINIA, Brampton (CA); Mohammad Reza REZAEI, Toronto (CA); Navid ZIAEI, North York (CA); Mehdi AARABI, Richmond Hill (CA); Reza SAADATI FARD, Worcester, MA (US); Saba RAHIMI, Thornhill (CA); Amirmohammad SAMIEZADEH, North York (CA); Pouria TAVAKKOLI AVVAL, Richmond Hill (CA); Kathryn TINCKAM, Toronto (CA); Darren YUEN, North York (CA); Sang Joseph KIM, Toronto (CA); Nazia SELZNER, North York (CA); Darin TRELEAVEN, Hamilton (CA); Pouyan SHAKER, Toronto (CA); Mansour ABOLGHASEMIAN, North York (CA)

(73) Assignee: ORTHO BIOMED INC., Toronto, ON (CA)

(21) Appl. No.: 18/721,680

(22) PCT Filed: Jan. 10, 2022
(86) PCT No.: PCT/IB2022/050132

§ 371 Date: Jun. 19, 2024

(51) Int. Cl. G16H 20/40; G16H 10/60; G16H 50/30; G16H 50/70

(52) U.S. Cl. CPC G16H 20/40; G16H 10/60; G16H 50/30; G16H 50/70

(57) ABSTRACT

A method for identifying a plurality of intended organ donors among a plurality of organ donor candidates. The method includes obtaining a donor clinical dataset by acquiring each donor clinical data from a respective organ donor candidate, obtaining a recipient clinical dataset by acquiring each recipient clinical data from a respective recipient candidate, predicting one of an in-hospital death or survival of an intended organ donor candidate, estimating a time of death of the intended organ donor candidate, obtaining a paired donor-recipient by pairing the intended organ donor candidate with an intended recipient for organ transplantation, estimating a probability of organ transplant success for the paired donor-recipient, and pairing the intended recipient with the plurality of intended organ donors for organ transplantation based on the probability of organ transplant success.

### TECHNICAL FIELD

The present disclosure generally relates to survival analysis, and particularly, to organ transplantation prognosis.

### BACKGROUND ART

Organ transplantation is a process of removing a biological organ from a donor's body and replacing it with a damaged or missing organ in a recipient's body. It has been rapidly growing since its emergence, saving thousands of patients' lives. However, healthcare systems still face challenging issues for successful organ transplantation. An ongoing issue is a successful matchmaking between organ donors and recipients so that recipients receive appropriate organs at appropriate times. To achieve this goal, potential organ donors should be matched with proper recipients before a fatal damage occurs to vital organs of recipients.

Several studies have been conducted on donor-to-recipient matchmaking. For example, Grady et al. disclosed in U.S. Pat. No. 10,499,990 methods for assessing organ transplantation. Campagne et al. disclosed in U.S. Pat. No. 10,720,226 a method for organ matchmaking. Wohlgemuth et al. disclosed in U.S. Pat. No. 7,235,358 methods for monitoring transplant rejection. However, such methods mainly focus on recipients and attempt to find appropriate organ donors based on quality and time of transplantation. This approach may lead to challenges in finding appropriate donors in due time because of unbalanced number of organ donors and recipients (number of potential donors are usually smaller than recipients). Current healthcare systems lack a comprehensive strategy to pair potential organ donors with appropriate recipients.

There is, therefore, a need for a method that may be capable of identifying appropriate organ donors for organ transplantation in due time. There is further a need for a method that may predict success or failure of organ transplantation from potential organ donors to recipients. There is also a need for a method that may pair suitable organ donors to recipients based on organ transplantation predictions in due time.

### SUMMARY OF THE DISCLOSURE

This summary is intended to provide an overview of the subject matter of this patent, and is not intended to identify essential elements or key elements of the subject matter, nor is it intended to be used to determine the scope of the claimed implementations. The proper scope of this patent may be ascertained from the claims set forth below in view of the detailed description below and the drawings.

In one general aspect, the present disclosure describes an exemplary method for identifying a plurality of intended organ donors among a plurality of organ donor candidates based on artificial intelligence. An exemplary method may include obtaining a donor clinical dataset by acquiring each donor clinical data in the donor clinical dataset from a respective organ donor candidate of the plurality of organ donor candidates that may be hospitalized in an intensive care unit (ICU), obtaining a recipient clinical dataset by acquiring each recipient clinical data in the recipient clinical dataset from a respective recipient candidate of a plurality of recipient candidates, predicting one of an in-hospital death or survival of an intended organ donor candidate of the plurality of organ donor candidates based on intended donor clinical data in the donor clinical dataset, estimating a time of death of the intended organ donor candidate responsive to the in-hospital death of the intended organ donor candidate being predicted, obtaining a paired donor-recipient by pairing the intended organ donor candidate with an intended recipient of the plurality of recipient candidates for organ transplantation based on the intended donor clinical data and the recipient clinical dataset responsive to the time of death being in a predefined time period, estimating a probability of organ transplant success for the paired donor-recipient based on the intended donor clinical data and intended recipient clinical data in the recipient clinical dataset, and pairing the intended recipient with the plurality of intended organ donors for organ transplantation based on the probability of organ transplant success. An exemplary intended donor clinical data may be acquired from the intended organ donor candidate. An exemplary intended recipient clinical data may be acquired from the intended recipient.

In an exemplary embodiment, each of predicting the one of the in-hospital death or the survival of the intended organ donor candidate and estimating the time of death may include generating a gated recurrent unit with trainable decays (GRU-D) output from the intended donor clinical data by applying the intended donor clinical data to a GRU-D layer, generating a hidden state from the GRU-D output by applying the GRU-D output to a recurrent neural network (RNN), generating a latent variable from the hidden state, and generating one of a classification output or a regression output by applying an activation function to the latent variable. In an exemplary embodiment, the GRU-D layer and the RNN may be associated with a GRU-D neural network. An exemplary GRU-D neural network may include a Bayesian neural network. An exemplary RNN may include a plurality of RNN layers. An exemplary classification output may include the one of the in-hospital death or the survival. An exemplary regression output may include the time of death.

In an exemplary embodiment, generating the latent variable from the hidden state may include generating a first (1^{st}) dense output of a plurality of dense outputs from the hidden state by feeding the hidden state to a first (1^{st}) dense layer of a plurality of dense layers, generating a first (1^{st}) dropout output of a plurality of dropout outputs by applying a dropout process on the 1^{st} dense output, generating an n^{th} dense output of the plurality of dense outputs from an (n−1)^{th} dropout output of the plurality of dropout outputs by feeding the (n−1)^{th} dropout output to an n^{th} dense layer of the plurality of dense layers where 1<n≤N_{d} and N_{d} is a number of the plurality of dense layers, and generating an n^{th} dropout output of the plurality of dropout outputs from the n^{th} dense output by applying the dropout process on the n^{th} dense output. An exemplary plurality of dense layers may be associated with the GRU-D neural network. An exemplary N_{d}^{th} dropout output of the plurality of dropout outputs may include the latent variable.

In an exemplary embodiment, applying the activation function to the latent variable may include applying a sigmoid function to the latent variable. In an exemplary embodiment, applying the activation function to the latent variable may include applying a rectified linear unit (ReLU) function to the latent variable.

In an exemplary embodiment, estimating the time of death may further include estimating a probability density function (PDF) of the time of death by generating a gated recurrent unit with trainable decays (GRU-D) output from the intended donor clinical data by applying the intended donor clinical data to a GRU-D layer, generating an encoded sequence from the GRU-D output by applying the GRU-D output to a first recurrent neural network (RNN), generating a decoded sequence from the encoded sequence by applying the encoded sequence to a second RNN, generating an event-related sequence from the encoded sequence by applying an attention mechanism on the encoded sequence based on the decoded sequence, generating a concatenated sequence by concatenating the event-related sequence and the decoded sequence, and generating the PDF of the time of death from the concatenated sequence by applying the concatenated sequence to a time distributed dense layer. In an exemplary embodiment, the GRU-D layer, the first RNN, the second RNN, and the time distributed dense layer may be associated a sequence-to-sequence (seq2seq) neural network. An exemplary seq2seq neural network may include a Bayesian neural network. An exemplary first RNN may include a first plurality of RNN layers. An exemplary second RNN may include a second plurality of RNN layers. In an exemplary embodiment, the decoded sequence and the event-related sequence may be associated with the time of death.

In an exemplary embodiment, pairing the intended organ donor candidate with the intended recipient may include training the seq2seq neural network by minimizing a reverse loss function based on the ICU dataset, extracting a donor feature set from the intended donor clinical data utilizing the seq2seq neural network by applying the intended donor clinical data to the GRU-D layer, extracting each of a plurality of recipient feature sets from a respective recipient clinical data in the recipient clinical dataset utilizing the seq2seq neural network by applying the respective recipient clinical data to the GRU-D layer, grouping the donor feature set and a subset of the plurality of recipient feature sets in a donor cluster of a plurality of clusters by clustering the donor feature set and the plurality of recipient feature sets into a plurality of clusters based on distances between different feature sets among the donor feature set and the plurality of recipient feature sets, obtaining a plurality of mean squared errors (MSEs) by calculating MSEs between the donor feature set and each of the plurality of recipient feature sets in the subset, finding a smallest MSE among the plurality of MSEs, and pairing the intended organ donor candidate with a most similar recipient candidate of the plurality of recipient candidates to the intended organ donor candidate. An exemplary most similar recipient candidate may be associated with a most similar recipient feature set of the plurality of recipient feature sets in the subset to the donor feature set. An exemplary most similar recipient feature set may be associated with the smallest MSE.

In an exemplary embodiment, estimating the probability of the organ transplant success for the paired donor-recipient may include estimating a plurality of probability density functions (PDFs) for a plurality of events for the paired donor-recipient. An exemplary plurality of events may be associated with the organ transplant success. In an exemplary embodiment, estimating the plurality of PDFs for the plurality of events may include estimating each respective PDF of the plurality of PDFs for one of death time of the intended recipient, a first graft failure due to early-onset pathologies (EOPs) of the intended recipient, a second graft failure due to late-onset pathologies (LOPs) of the intended recipient, a third graft failure due to acute rejection of the intended recipient's body, a fourth graft failure due to chronic rejection of the intended recipient's body, and a fifth graft failure due to other causes.

In an exemplary embodiment, estimating the plurality of PDFs may include generating a first (1^{st}) dense output of a plurality of dense outputs from the intended donor clinical data and the intended recipient clinical data by applying the intended donor clinical data and the intended recipient clinical data to a first (1^{st}) dense layer of a plurality of dense layers, generating a first (1^{st}) dropout output of a plurality of dropout outputs by applying a dropout process to the 1^{st} dense output, generating an m^{th} dense output of the plurality of dense outputs from an (m−1)^{th} dropout output of the plurality of dropout outputs by applying the (m−1)^{th} dropout output to an m^{th} dense layer of the plurality of dense layers where 1<m≤M_{d} and Ma is a number of the plurality of dense layers, generating an m^{th} dropout output of the plurality of dropout outputs from the m^{th} dense output by applying the dropout process to the m^{th} dense output, generating a normalized output by applying a batch normalization process to the an M_{d}^{th} dropout output of the plurality of dropout outputs, generating a plurality of cause-specific outputs from the normalized output, the intended donor clinical data, and the intended recipient clinical data by applying the normalized output, the intended donor clinical data, and the intended recipient clinical data to a plurality of cause-specific subnetworks, generating a concatenated sequence by concatenating the plurality of cause-specific outputs, and generating each of the plurality of PDFs for each respective event of the plurality of events from the concatenated sequence by applying the concatenated sequence to a time distributed dense layer.

In an exemplary embodiment, the plurality of dense layers and the plurality of cause-specific subnetworks may be associated with a one-to-many (one2seq) neural network. An exemplary one2seq neural network may include a Bayesian neural network. In an exemplary embodiment, each of the plurality of cause-specific subnetworks may include a respective plurality of gated recurrent unit (GRU) layers.

In an exemplary embodiment, pairing the intended recipient with the plurality of intended organ donors may include training a sequence-to-sequence (seq2seq) neural network by minimizing a reverse loss function based on the ICU dataset, extracting a recipient feature set from the intended recipient clinical data utilizing the seq2seq neural network by applying the intended recipient clinical data to the seq2seq neural network, extracting each of a plurality of donor feature sets from a respective donor clinical data in the donor clinical dataset utilizing the seq2seq neural network by applying the respective donor clinical data to the seq2seq neural network, grouping the recipient feature set and a subset of the plurality of donor feature sets in a recipient cluster of a plurality of clusters by clustering the recipient feature set and the plurality of donor feature sets into a plurality of clusters based on distances between different feature sets among the recipient feature set and the plurality of donor feature sets, obtaining a plurality of mean squared errors (MSEs) by calculating MSEs between the recipient feature set and each of the plurality of donor feature sets in the subset, extracting an MSE subset from the plurality of MSEs, extracting an organ donor candidates subset from the plurality of organ donor candidates, and pairing the intended recipient with each organ donor candidate in the organ donor candidates subset. In an exemplary embodiment, each MSE in the MSE subset may include a value smaller than an MSE threshold. Each exemplary organ donor candidate in the organ donor candidates subset may be associated with a respective MSE in the MSE subset.

In an exemplary embodiment, each of extracting the recipient feature set by applying the intended recipient clinical data to the seq2seq neural network and extracting each of the plurality of donor feature sets by applying the respective donor clinical data to the seq2seq neural network may include estimating a plurality of probability density functions (PDFs) for a plurality of events from input data. An exemplary input data may include one of the intended recipient clinical data or the respective donor clinical data. An exemplary plurality of events may be associated with one of the intended recipient or a respective organ donor candidate of the plurality of organ donor candidates. In an exemplary embodiment, the plurality of events may include death time, a first graft failure due to early-onset pathologies (EOPs), a second graft failure due to late-onset pathologies (LOPs), a third graft failure due to acute rejection, a fourth graft failure due to chronic rejection, and a fifth graft failure due to other causes.

In an exemplary embodiment, estimating the plurality of PDFs may include generating a gated recurrent unit with trainable decays (GRU-D) output from the input data by applying the input data to a GRU-D layer, generating an encoded sequence from the GRU-D output by applying the GRU-D output to an encoder recurrent neural network (RNN), generating a plurality of decoded sequences from the encoded sequence by applying the encoded sequence to a plurality of decoder RNNs, generating a plurality of event-related sequences from the encoded sequence by applying an attention mechanism to the encoded sequence based on a respective decoded sequence of the plurality of decoded sequences, generating a plurality of concatenated sequences by concatenating each of the plurality of event-related sequences and a respective decoded sequence of the plurality of decoded sequences, and generating each of the plurality of PDFs for each respective event of the plurality of events from a respective concatenated sequence of the plurality of concatenated sequences by applying each of the plurality of concatenated sequences to a respective time distributed dense layer. In an exemplary embodiment, the GRU-D layer, the encoder RNN, and the plurality of decoder RNNs may be associated with the seq2seq neural network. An exemplary encoder RNN may include a first plurality of RNN layers. In an exemplary embodiment, each of the plurality of decoder RNNs may include a respective second plurality of RNN layers.

Other exemplary systems, methods, features and advantages of the implementations will be, or will become, apparent to one of ordinary skill in the art upon examination of the following figures and detailed description. It is intended that all such additional systems, methods, features and advantages be included within this description and this summary, be within the scope of the implementations, and be protected by the claims herein.

### BRIEF DESCRIPTION OF THE DRAWINGS

The drawing figures depict one or more implementations in accord with the present teachings, by way of example only, not by way of limitation. In the figures, like reference numerals refer to the same or similar elements.

FIG. 1A shows a flowchart of a method for identifying a plurality of intended organ donors among a plurality of organ donor candidates based on artificial intelligence, consistent with one or more exemplary embodiments of the present disclosure.

FIG. 1B shows a flowchart for each of predicting one of in-hospital death or survival of an intended organ donor candidate and estimating a time of death, consistent with one or more exemplary embodiments of the present disclosure.

FIG. 1C shows a flowchart for generating a latent variable from a hidden state, consistent with one or more exemplary embodiments of the present disclosure.

FIG. 1D shows a flowchart of a method for estimating a probability density function (PDF) of a time of death of an intended organ donor candidate, consistent with one or more exemplary embodiments of the present disclosure.

FIG. 1E shows a flowchart of a method for pairing an intended organ donor candidate with an intended recipient, consistent with one or more exemplary embodiments of the present disclosure.

FIG. 1F shows a flowchart of a method for estimating a plurality of PDFs for a plurality of events associated with a paired donor-recipient, consistent with one or more exemplary embodiments of the present disclosure.

FIG. 1G shows a flowchart for generating a latent variable from intended donor clinical data and intended recipient clinical data, consistent with one or more exemplary embodiments of the present disclosure.

FIG. 1H shows a flowchart for pairing an intended recipient with a plurality of intended organ donors, consistent with one or more exemplary embodiments of the present disclosure.

FIG. 1I shows a flowchart of a method for estimating a plurality of PDFs for a plurality of events associated with an intended recipient or an organ donor candidate, consistent with one or more exemplary embodiments of the present disclosure.

FIG. 2A shows a block diagram of a system for identifying a plurality of intended organ donors among a plurality of organ donor candidates based on artificial intelligence, consistent with one or more exemplary embodiments of the present disclosure.

FIG. 2B shows a block diagram of a gated recurrent unit with trainable decays (GRU-D) neural network, consistent with one or more exemplary embodiments of the present disclosure.

FIG. 2C shows a block diagram of a dense network for generating a latent variable from a hidden state, consistent with one or more exemplary embodiments of the present disclosure.

FIG. 2D shows a block diagram of a sequence-to-sequence (seq2seq) neural network for time of death estimation, consistent with one or more exemplary embodiments of the present disclosure.

FIG. 2E shows a block diagram of a one-to-many (one2seq) neural network, consistent with one or more exemplary embodiments of the present disclosure.

FIG. 2F shows a block diagram of a dense network for generating a latent variable from intended donor clinical data and intended recipient clinical data, consistent with one or more exemplary embodiments of the present disclosure.

FIG. 2G shows a block diagram of a seq2seq neural network for pairing an intended recipient with a plurality of intended organ donors, consistent with one or more exemplary embodiments of the present disclosure.

FIG. 3A shows a schematic of a plurality of clusters for grouping a donor feature set and a subset of a plurality of recipient feature sets, consistent with one or more exemplary embodiments of the present disclosure.

FIG. 3B shows a schematic of a donor cluster, consistent with one or more exemplary embodiments of the present disclosure.

FIG. 4A shows a schematic of a plurality of clusters for grouping a recipient feature set and a subset of a plurality of donor feature sets, consistent with one or more exemplary embodiments of the present disclosure.

FIG. 4B shows a schematic of a recipient cluster, consistent with one or more exemplary embodiments of the present disclosure.

FIG. 5 shows a high-level functional block diagram of a computer system, consistent with one or more exemplary embodiments of the present disclosure.

FIG. 6 shows a PDF, a cumulative distribution function (CDF), and an expected time of death predicted by a seq2seq neural network, consistent with one or more exemplary embodiments of the present disclosure.

FIG. 7 shows error distribution of a one2seq neural network and a seq2seq neural network, consistent with one or more exemplary embodiments of the present disclosure.

### DESCRIPTION OF EMBODIMENTS

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

Herein is disclosed an exemplary method for identifying appropriate organ donors (i.e., intended organ donors) among potential organ donors (i.e., organ donor candidates) for organ transplantation to one or more intended recipients. An exemplary method may analyze clinical data of potential donors who are hospitalized in an intensive care unit (ICU). An exemplary method may predict in-hospital death probability of such patients and may estimate their death time if in-hospital death of exemplary patients is predicted. Based on clinical data and estimated death time of an exemplary organ donor, an exemplary recipient (i.e., intended recipient) may be identified among a number of potential recipients that may be in need for organ transplantation. An exemplary intended recipient may be more similar to an exemplary intended organ donor than other potential recipients in terms of estimated death time. An exemplary method may proceed to estimating probability distribution of several failures due to organ transplantation (i.e., graft failure) to an exemplary intended recipient. Based on estimated probability distributions, a group of intended organ donors may be identified among potential donors that may be more similar to an exemplary intended recipient than other potential donors in terms of exemplary probability distributions. An exemplary method may measure similarity by estimating probability distribution of graft failures similar to those of the intended recipient and comparing estimated distributions for potential donors with corresponding ones for the intended recipient. An exemplary group of intended organ donors may be paired with an exemplary intended recipient for possible organ transplantation. An exemplary method may utilize different artificial neural network structures for implementing different steps of the method.

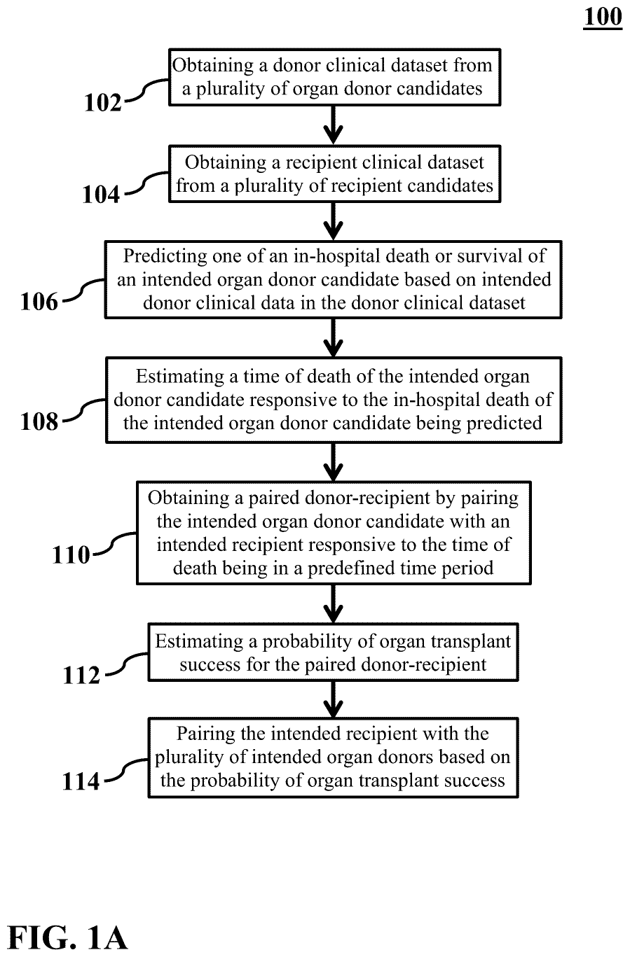

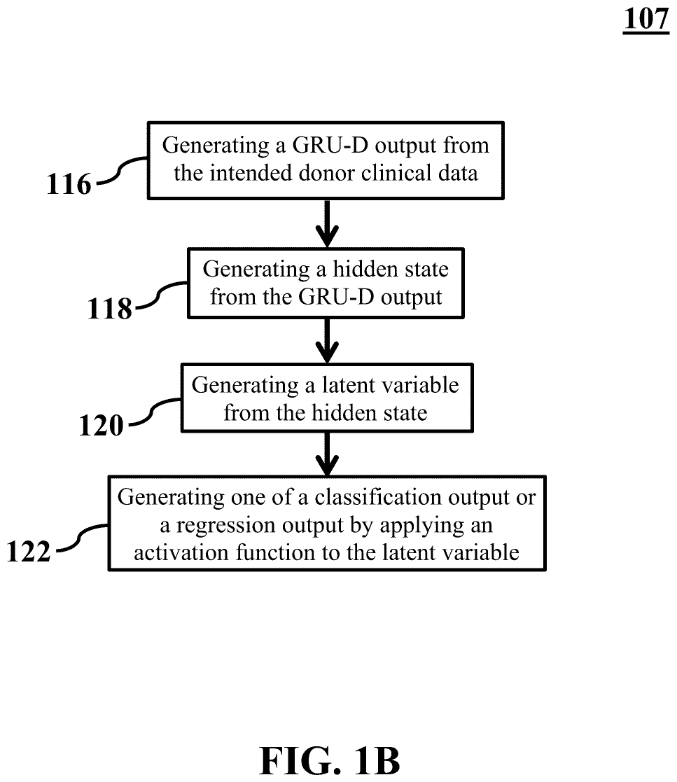

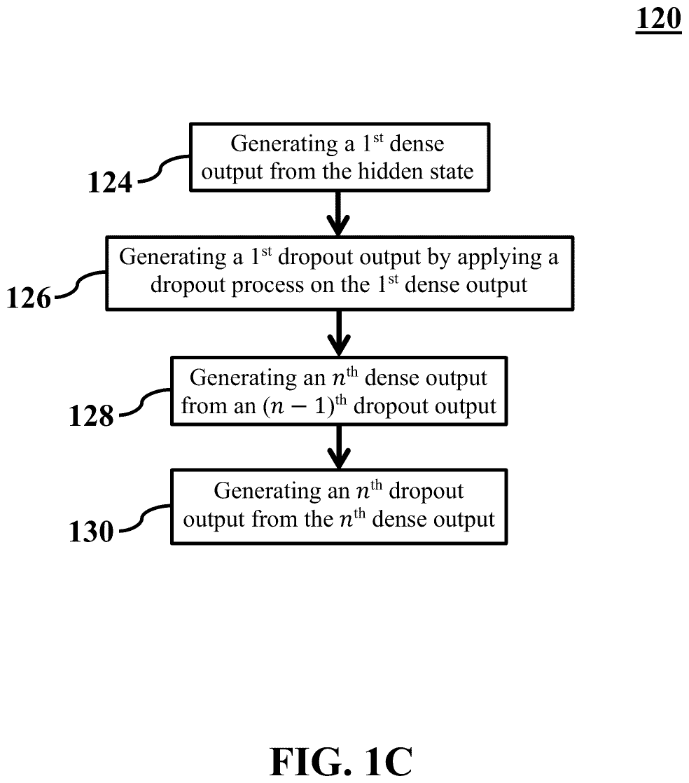

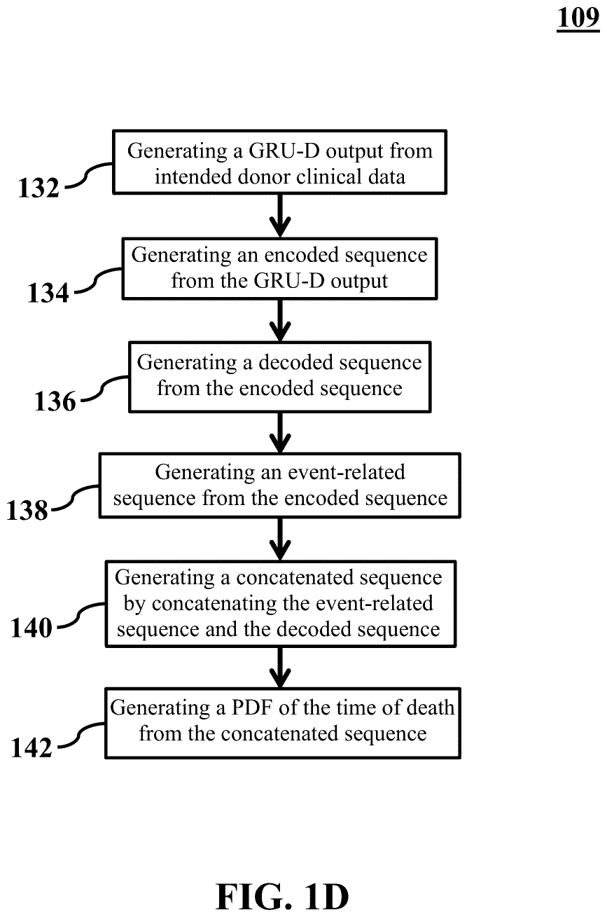

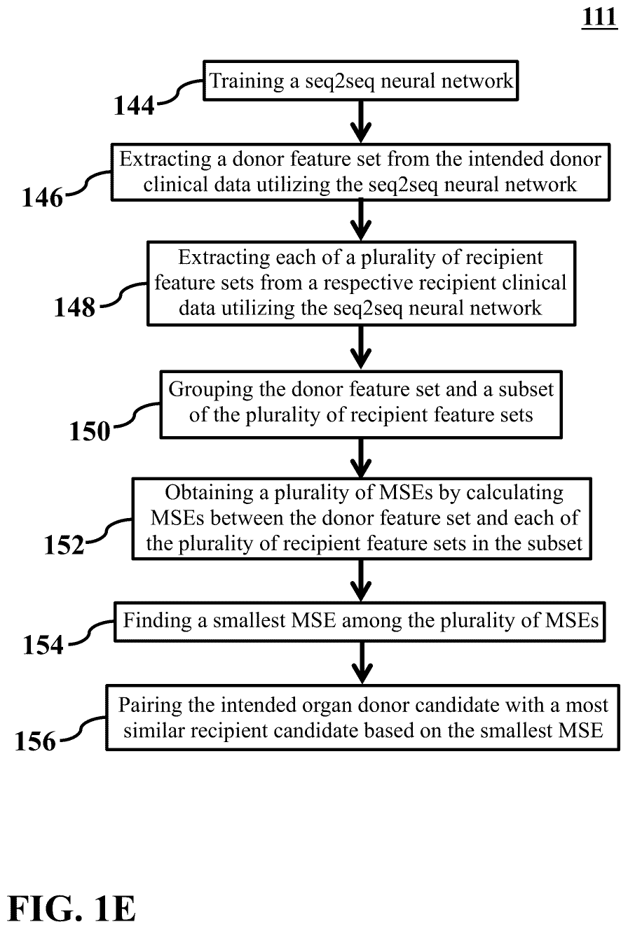

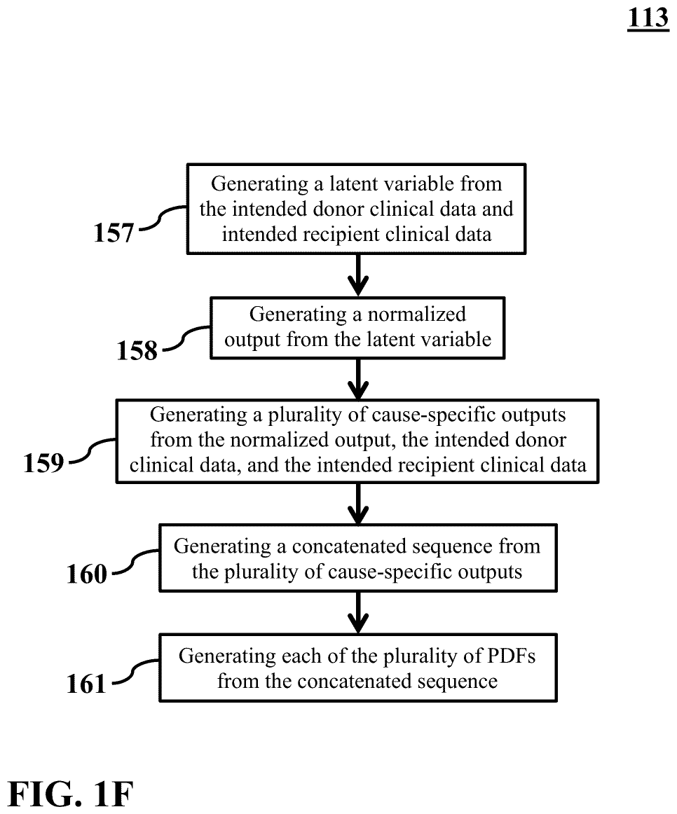

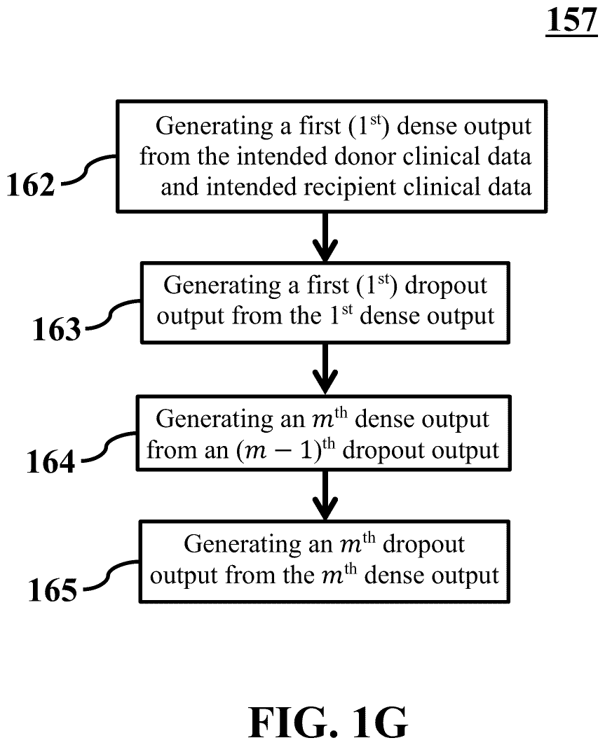

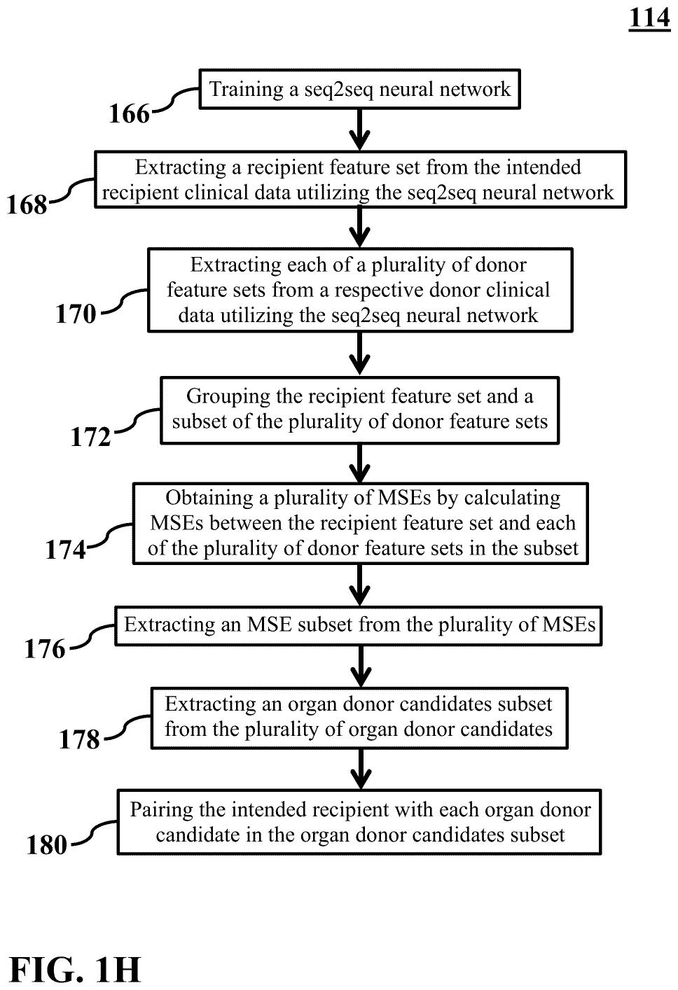

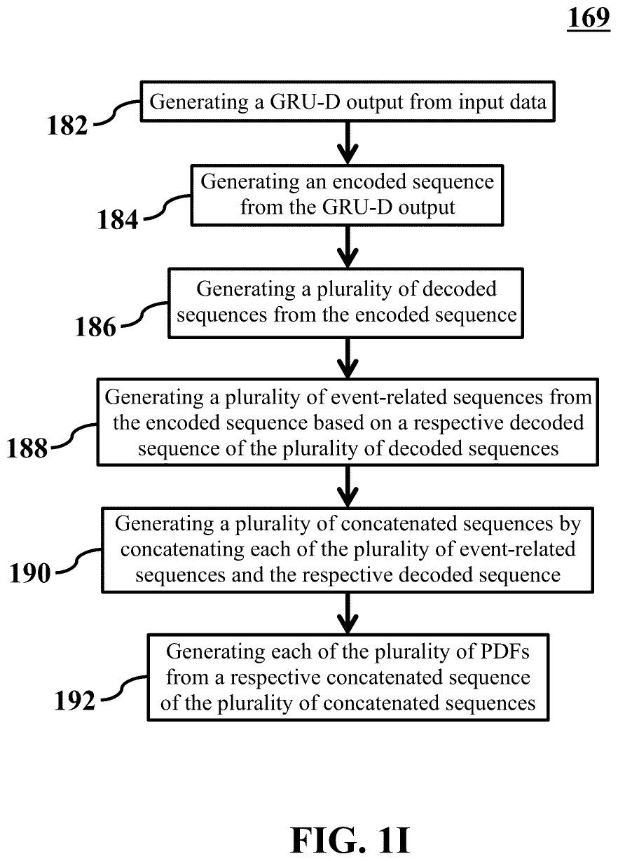

FIG. 1A shows a flowchart of a method for identifying a plurality of intended organ donors among a plurality of organ donor candidates based on artificial intelligence, consistent with one or more exemplary embodiments of the present disclosure. An exemplary method 100 may include obtaining a donor clinical dataset from a plurality of organ donor candidates (step 102), obtaining a recipient clinical dataset from a plurality of recipient candidates (step 104), predicting one of an in-hospital death or survival of an intended organ donor candidate of the plurality of organ donor candidates based on intended donor clinical data in the donor clinical dataset (step 106), estimating a time of death of the intended organ donor candidate responsive to the in-hospital death of the intended organ donor candidate being predicted (step 108), obtaining a paired donor-recipient by pairing the intended organ donor candidate with an intended recipient of the plurality of recipient candidates for organ transplantation based on the intended donor clinical data and the recipient clinical dataset responsive to the time of death being in a predefined time period (step 110), estimating a probability of organ transplant success for the paired donor-recipient (step 112), and pairing the intended recipient with the plurality of intended organ donors for organ transplantation based on the probability of organ transplant success (step 114).

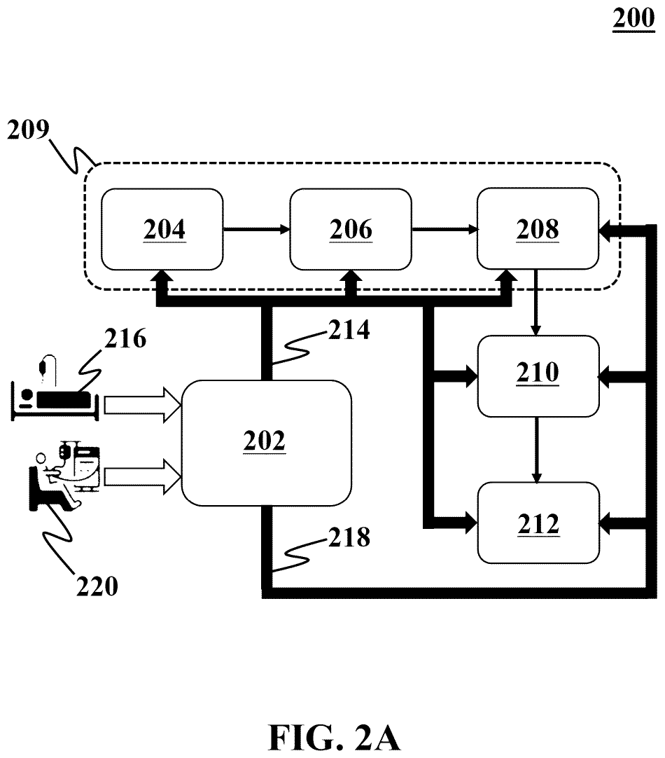

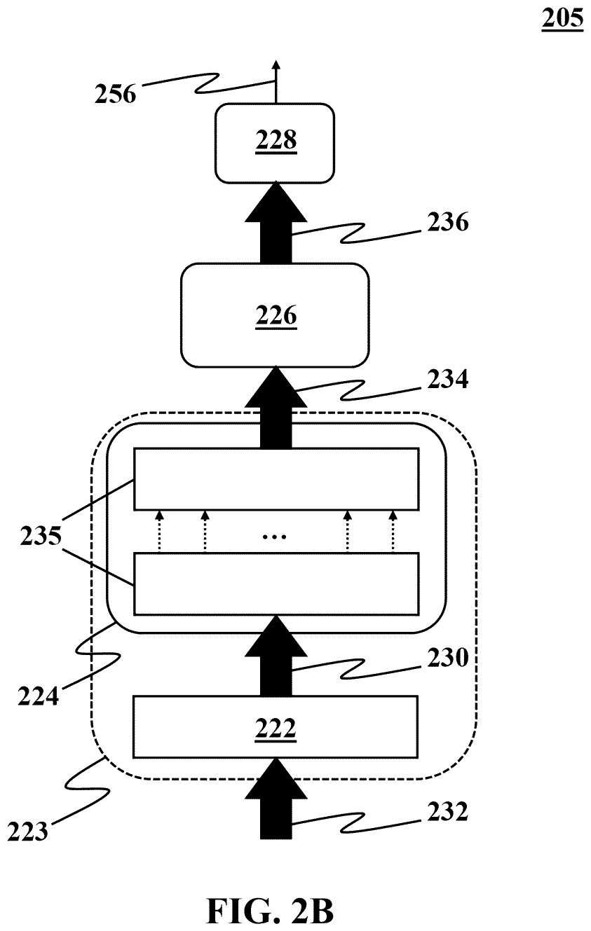

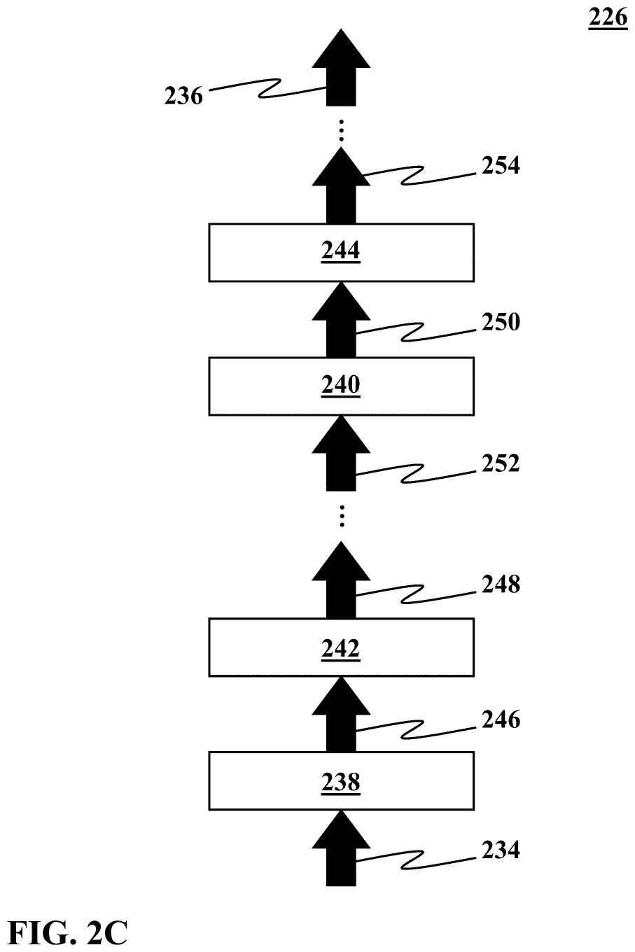

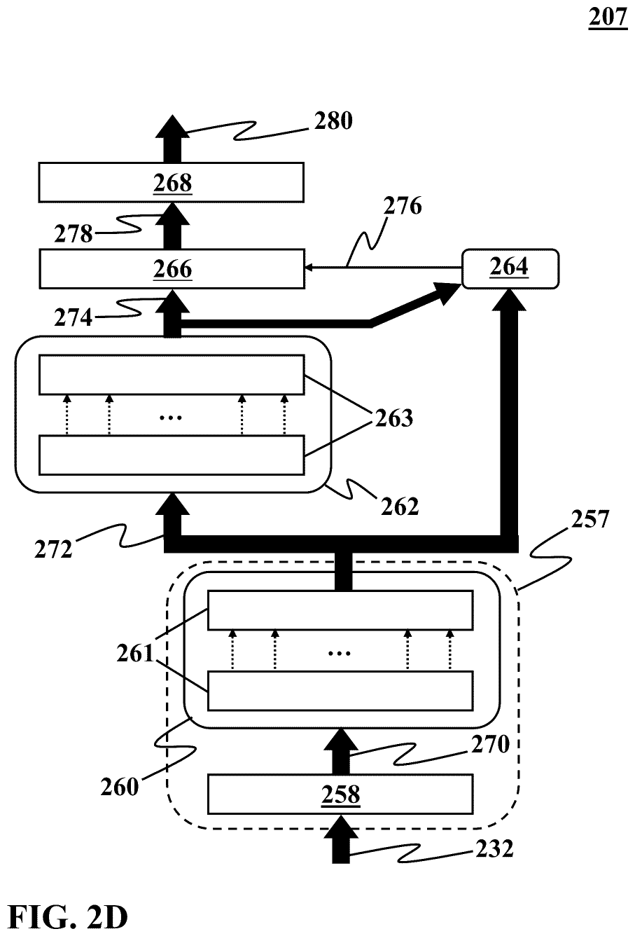

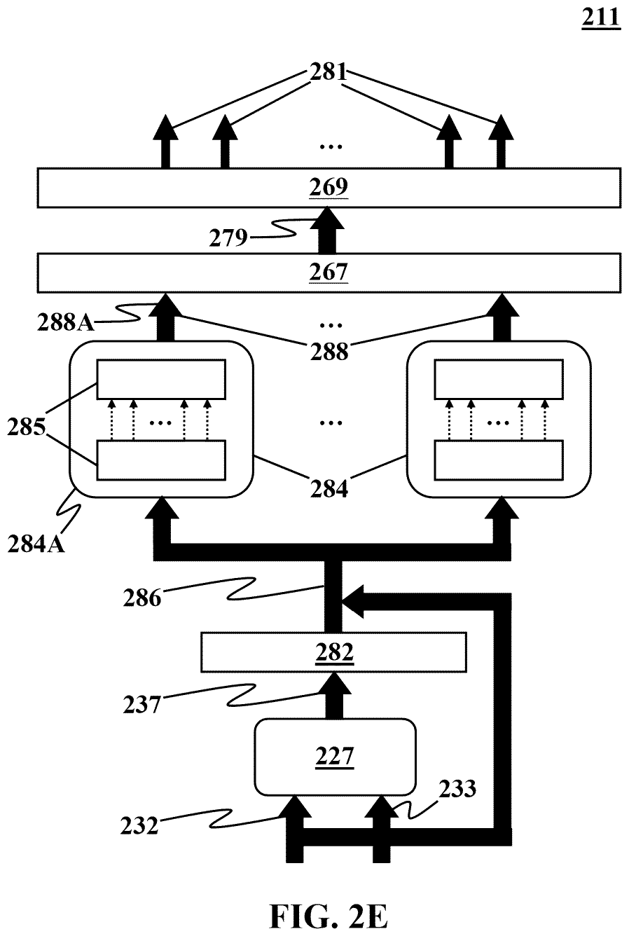

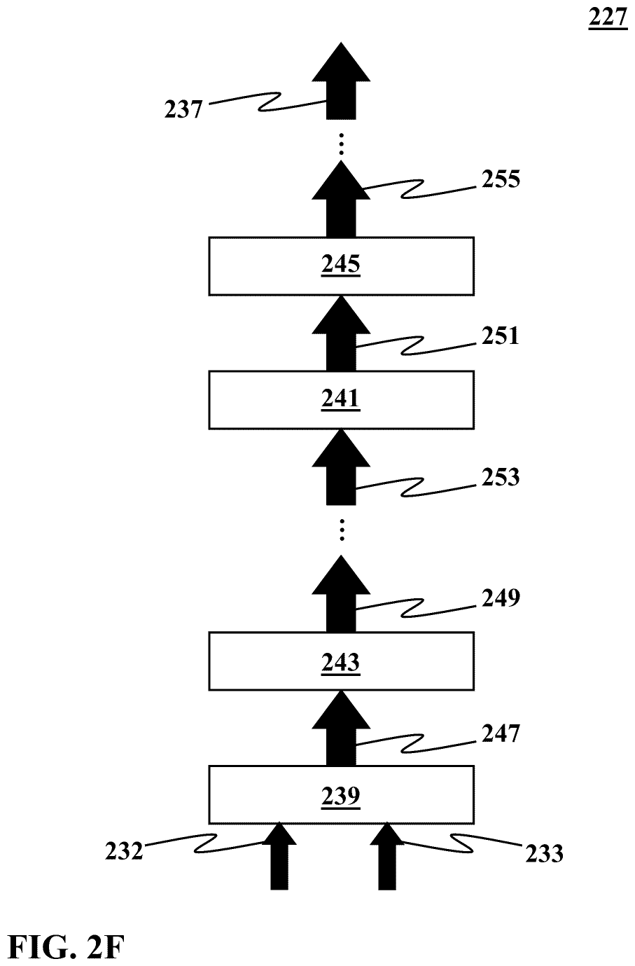

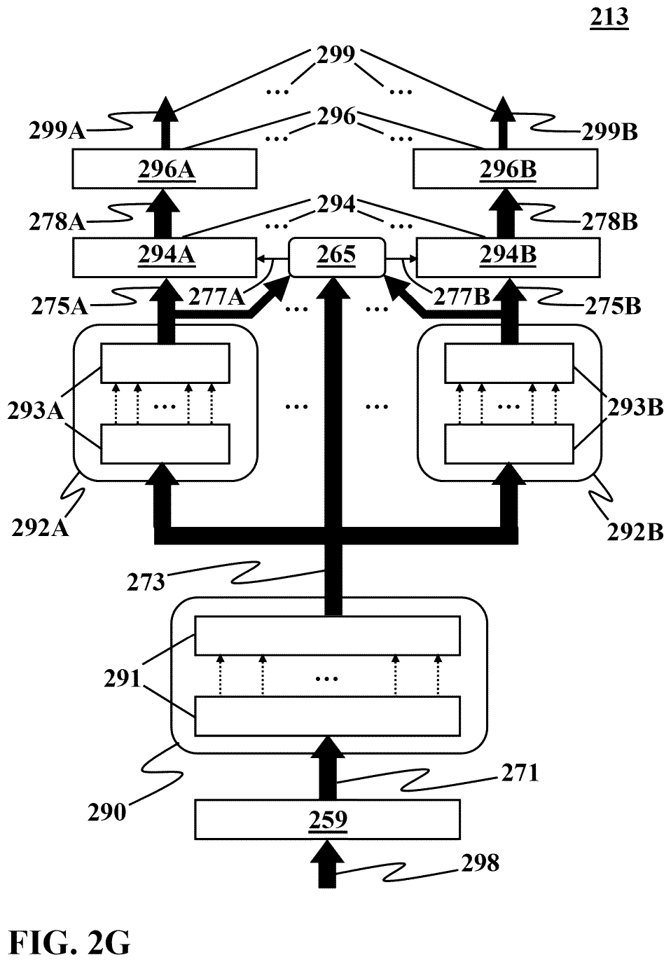

FIG. 2A shows a block diagram of a system for identifying a plurality of intended organ donors among a plurality of organ donor candidates based on artificial intelligence, consistent with one or more exemplary embodiments of the present disclosure. In an exemplary embodiment, different steps of method 100 may be implemented utilizing a system 200. In an exemplary embodiment, system 200 may include a data acquisition unit 202, a prediction block 204, an estimation block 206, a donor-to-recipient pairing block 208, an organ match making and monitoring (OMM) block 210, and a recipient-to-donor pairing block 212.

In an exemplary embodiment, an ensemble of prediction block 204, estimation block 206, and donor-to-recipient pairing block 208 may be referred to a donation after circulatory death (DCD) module 209. In an exemplary embodiment, DCD module 209 may utilize prediction block 204 to predict if a patient that is hospitalized in an intensive care unit (ICU) may die or may survive the current ICU stay. DCD module 209 may also predict probability and time of death of an exemplary ICU patient utilizing estimation block 206 if prediction block 204 predicts death of the ICU patient. Transplant authorities may use exemplary predicted time of death to prepare for organ harvest and transplant. In an exemplary embodiment, probability and time of death of the ICU patient may be referred to as death candidacy indicators (DCI) of the ICU patient. An exemplary DCI of an ICU patient (i.e., a donor) may be used to provide a list of potential patients (i.e., recipients) for organ harvesting and their predicted time of death, so that healthcare professionals may proceed to preparing such patients for organ harvesting and performing legal protocols for transplantation. As a result, quantity and quality of donations after circulatory death may be improved. In an exemplary embodiment, DCD module 209 may utilize donor-to-recipient pairing block 208 (also called reverse DCD block) to produce justified pairings of potential donors with potential recipients based on predictions of prediction block 204 and estimation block 206, so that physicians may become confident about accuracy and reliability of predictions. As a result, a valuable means may be provided for healthcare professionals to obtain individualized confidence intervals for each prediction per patient, to contemplate observations from patients from a dataset used by system 200 as a basis for its predictions, and to identify covariates (i.e., patient characteristics) with highest impact for each outcome.

In an exemplary embodiment, OMM block 210 may calculate probability of transplant success of different organs to potential recipients based on physiological, immunological, and demographic data of potential recipients and donors. In an exemplary embodiment, OMM block 210 may predict longevity of an offered organ if transplanted, and also an expected survivorship of a recipient. Exemplary OMM output data may be presented to a physician for improving the quality of matchmaking between potential recipients and donors. If an organ is transplanted, an exemplary recipient may also be monitored by OMM block 210 based on a combination of pre-graft data in addition to post-graft clinical, physiological and therapeutic data of the recipient after transplantation for monitoring the prognosis of the transplant. Data from post-transplant monitoring may be used to improve future predictions. In an exemplary embodiment, recipient-to-donor pairing block 212 may present potential donors that may be similar to the recipient for more informed decision making. In an exemplary embodiment, OMM block 210 may predict a risk of early failure (for example, organ failure within a year of an organ transplant), survivorship (longevity) of a graft with a potential recipient, and life expectancy of a potential recipient after receiving a certain graft.

For further detail with respect to step 102, in an exemplary embodiment, obtaining a donor clinical dataset 214 may include acquiring each donor clinical data in donor clinical dataset 214 from a respective organ donor candidate (for example, an intended organ donor candidate 216) of a plurality of organ donor candidates that may be hospitalized in an ICU. Exemplary intended donor clinical data may be acquired from intended organ donor candidate 216. In an exemplary embodiment, the intended donor clinical data may include age, gender, height, type (deceased vs living), blood group, creatinine, history of diabetes or hypertension, and ischemic times of intended organ donor candidate 216. In an exemplary embodiment, data acquisition unit 202 may be utilized for obtaining clinical data from each organ donor candidate. In an exemplary embodiment, data acquisition unit 202 may include different data acquisition devices such as medical imaging modalities (for example, ultrasound, magnetic resonance, computed tomography, etc.) and biomedical sensors that may allow for measuring different biomedical signals (for example, electrocardiogra (ECG) or electroencephalography (EEG) electrodes) or physiological parameters (for example, blood pressure, Oxygen level, heart rate, etc.). Different types of clinical data may be acquired by data acquisition unit 202, for example, vital signs, administered fluids, laboratory measurements, microbiology information, excreted fluids, and prescriptions.

In further detail with regards to step 104, in an exemplary embodiment, obtaining a recipient clinical dataset 218 may include acquiring each recipient clinical data in recipient clinical dataset 218 from a respective recipient candidate (for example, an intended recipient 220) of a plurality of recipient candidates. Exemplary intended recipient clinical data may be acquired from intended recipient 220. In an exemplary embodiment, the intended recipient clinical data may include height, weight, panel reactive antibody, and histocompatibility features of intended recipient 220. In an exemplary embodiment, data acquisition unit 202 may be utilized for obtaining clinical data from each recipient candidate, similar to obtaining clinical data from organ donor candidates, as described above in step 102.

In an exemplary embodiment, step 106 may include predicting one of an in-hospital death or survival of intended organ donor candidate 216 based on the intended donor clinical data utilizing prediction block 204. If, in an exemplary embodiment, in-hospital death of intended organ donor candidate 216 is predicted by prediction block 204, method 100 may proceed to step 108 to estimate a time of death of intended organ donor candidate 216 utilizing estimation block 206.

In further detail regarding steps 106 and 108, FIG. 1B shows a flowchart of a method for each of predicting one of in-hospital death or survival of an intended organ donor candidate and estimating a time of death, consistent with one or more exemplary embodiments of the present disclosure. An exemplary method 107 may include an implementation of predicting one of the in-hospital death or the survival of intended organ donor candidate 216 in step 106 or estimating the time of death in step 108. In an exemplary embodiment, method 107 may include generating a gated recurrent unit with trainable decays (GRU-D) output from the intended donor clinical data (step 116), generating a hidden state from the GRU-D output (step 118), generating a latent variable from the hidden state (step 120), and generating one of a classification output or a regression output by applying an activation function to the latent variable (step 122).

FIG. 2B shows a block diagram of a GRU-D neural network, consistent with one or more exemplary embodiments of the present disclosure. In an exemplary embodiment, different steps of method 107 may be implemented utilizing a GRU-D neural network 205. In an exemplary embodiment, GRU-D neural network 205 may accept longitudinal measurements (i.e., measurements that are sequentially obtained over time) of patients in ICU and predict if they survive the ICU stay or not. Besides, GRU-D neural network 205 has the capability to impute not-missing-at-random data that are widely present in medical records. In an exemplary embodiment, GRU-D neural network 205 may include an implementation of prediction block 204 or estimation block 206. In an exemplary embodiment, GRU-D neural network 205 may include a GRU-D layer 222, a recurrent neural network (RNN) 224, a dense network 226, and an activation layer 228.

For further detail regarding step 116, in an exemplary embodiment, generating a GRU-D output 230 from intended donor clinical data 232 may include applying intended donor clinical data 232 to GRU-D layer 222. In an exemplary embodiment, GRU-D layer 222 may include an implementation of GRU-D disclosed by Che et al. [“Recurrent neural networks for multivariate time series with missing values.” Scientific reports 8, no. 1 (2018): 1-12]. An exemplary GRU-D layer is an extension of a GRU cell with the ability to effectively impute missing values. GRU-D uses a mechanism to learn during a training phase of system 200 how much to focus on previous measurements and how much to focus on a mean of a covariate to impute missing values of a covariate.

For further detail with respect to step 118, in an exemplary embodiment, generating a hidden state 234 from GRU-D output 230 may include applying GRU-D output 230 to RNN 224. An exemplary ensemble of GRU-D layer 222 and RNN 224 may be referred to as an encoder 223. In an exemplary embodiment, RNN 224 may include a plurality of RNN layers 235 for improving performance of encoder 223. In an exemplary embodiment, RNN 224 may sequentially generate hidden state 234 for each step of the prediction horizon by observing values of hidden state 234 that are generated at previous steps. As a result, a smooth and virtually spike-free output may be generated by RNN 224.

In an exemplary embodiment, step 120 may include generating a latent variable 236 from hidden state 234. In an exemplary embodiment, latent variable 236 may refer to a variable that is not directly observed in an output of GRU-D neural network 205 but may be inferred from the output since the output may be generated from latent variable 236, as discussed later in step 122.

In further detail with regards to step 120, FIG. 1C shows a flowchart for generating a latent variable from a hidden state, consistent with one or more exemplary embodiments of the present disclosure. Referring to FIGS. 1C and 2B, in an exemplary embodiment, generating latent variable 236 from hidden state 234 in step 120 may include generating a first (1^{st}) dense output of a plurality of dense outputs from the hidden state (step 124), generating a first (1^{st}) dropout output of a plurality of dropout outputs by applying a dropout process on the 1^{st} dense output (step 126), generating an n^{th} dense output of the plurality of dense outputs from an (n−1)^{th} dropout output of the plurality of dropout outputs (step 128), and generating an n^{th} dropout output of the plurality of dropout outputs from the n^{th} dense output (step 130).

FIG. 2C shows a block diagram of a dense network for generating a latent variable from a hidden state, consistent with one or more exemplary embodiments of the present disclosure. In an exemplary embodiment, different steps of flowchart 120 may be implemented utilizing dense network 226. In an exemplary embodiment, dense network 226 may include a plurality of dense layers and a plurality of dropout layers. Exemplary plurality of dense layers may include a first (1^{st}) dense layer 238 and an n^{th} dense layer 240 where 1<n≤N_{d} and N_{d} is a number of the plurality of dense layers. Neurons of each exemplary dense layer may be connected to every neuron of a preceding dense layer. An exemplary plurality of dropout layers may include a first (1^{st}) dropout layer 242 and an n^{th} dropout layer 244.

Referring to FIGS. 1C and 2C, in an exemplary embodiment, step 124 may include generating a first (1^{st}) dense output 246 of the plurality of dense outputs from hidden state 234 by applying hidden state 234 to 1^{st} dense layer 238. For further detail with respect to step 126, in an exemplary embodiment, generating a first (1^{st}) dropout output 248 of the plurality of dropout outputs may include applying 1^{st} dense output 246 to 1^{st} dropout layer 242. In an exemplary embodiment, 1^{st} dropout layer 242 may perform a dropout process on 1^{st} dense output 246 to prevent overfitting. An exemplary dropout process may eliminate one or more elements of 1^{st} dense output 246 in a training phase of dense network 226 with a predefined probability that may be adjusted such that a negative impact of overfitting is suppressed.

In further detail regarding step 128, in an exemplary embodiment, generating an n^{th} dense output 250 of the plurality of dense outputs from an (n−1)^{th} dropout output 252 of the plurality of dropout outputs may include applying (n−1)^{th} dropout output 252 to n^{th} dense layer 240.

In further detail with regards to step 130, in an exemplary embodiment, generating an n^{th} dropout output 254 of the plurality of dropout outputs from n^{th} dense output 250 may include applying n^{th} dense output 250 to n^{th} dropout layer 244. In an exemplary embodiment, n^{th} dropout layer 244 may perform a dropout process similar to the dropout process of step 126 on n^{th} dense output 250. An exemplary N_{d}^{th} dropout output of the plurality of dropout outputs may include latent variable 236.

Referring again to FIGS. 1B and 2B, in an exemplary embodiment, step 122 may include applying an exemplary activation function to latent variable 236. In an exemplary embodiment, activation layer 228 may apply the activation function to latent variable 236. An exemplary output 256 of activation layer 228 may include an exemplary classification output or an exemplary regression output. In an exemplary embodiment, to generate the classification output, applying the activation function to latent variable 236 may include applying a sigmoid function to latent variable 236. In an exemplary embodiment, a sigmoid function may refer to a mathematical function that has a characteristic sigmoid curve. An exemplary classification output may include in-hospital death or survival of intended organ donor candidate 216.

In an exemplary embodiment, to generate the regression output, applying the activation function to latent variable 236 may include applying a rectified linear unit (ReLU) function to latent variable 236. In an exemplary embodiment, a ReLU function may refer to a piecewise linear mathematical function that outputs its input directly if the input is positive and outputs zero otherwise. An exemplary regression output may include time of death of intended organ donor candidate 216.

Referring again to FIGS. 1A and 2B, in an exemplary embodiment, predicting in-hospital death or survival of intended organ donor candidate 216 in step 106 may include training GRU-D neural network 205 by minimizing a classification loss function based on an ICU dataset. An exemplary ICU dataset may include clinical data of patients that may have been hospitalized in ICU and have a known status of in-hospital death or survival. An exemplary classification loss function may be defined by the following:

$${L}_{classification} = {\sum }_{i = 1 , i ∈ {U}_{u}}^{{N}_{u}} {( {y}_{i , true}^{C} - {y}_{i , pred}^{C} )}^{2} / {N}_{u}$$

where L_{classification} is an exemplary classification loss function, Uᵤ is a set of uncensored data in the ICU dataset, Nᵤ is number of uncensored data in the set of uncensored data, y_{i,true}^{c} is ground truth data (i.e., death or survival of a patient in ICU used for training GRU-D neural network 205) for in-hospital death/survival classification of an i^{th} sample in the set of uncensored data, and y_{pred}^{c} is a predicted value for in-hospital death/survival classification of the i^{th} sample. In an exemplary embodiment, uncensored data may refer to data of patients that has been fully recorded during patients' stay in ICU.

For further detail with respect to step 108, in an exemplary embodiment, estimating the time of death of intended organ donor candidate 216 may include training GRU-D neural network 205 by minimizing a regression loss function based on the ICU dataset. To deal with imbalanced datasets (i.e., datasets with a different number of patients that survive an ICU stay from a number of patients that die in ICU), an exemplary weighted loss function may be used for training GRU-D neural network 205. Since the number of deceased patients in ICU is usually lower than alive patients, assigning a higher weight to dead patients in the loss function may allow for paying more attention to the dead cases, thereby increasing the quality of estimation for an imbalanced dataset. Therefore, an exemplary regression loss function may be defined by the following:

$${L}_{regression} = {\sum }_{i = 1 , i ∈ {U}_{u}}^{{N}_{u}} {( {y}_{i , true}^{r} - {y}_{i , pred}^{r} )}^{2} / {N}_{u} + κ {\sum }_{i = j , j ∈ {U}_{c}}^{{N}_{c}} max ( 0 , {y}_{j , pred}^{r} - {y}_{j , c}^{r} ) / {N}_{c}$$

where L_{regression} is an exemplary regression loss function, y_{i,true}^{r} is ground truth data for in-hospital time of death of an i^{th} uncensored sample in the set of uncensored data, y_{i,pred}^{r} is a predicted value for in-hospital time of death of the i^{th} uncensored sample, U_{c} is a set of censored data in the ICU dataset, N_{c} is number of censored data in the set of censored data, y_{j,pred}^{r} is a predicted value for in-hospital time of death of a j^{th} censored sample in the set of censored data, y_{j,c}^{r} is a censoring time of the j^{th} censored sample, and κ is a penalty coefficient. In an exemplary embodiment, censored data may refer to data of patients for which a medical center has lost track at some point in time (i.e., censoring time). Therefore, in an exemplary embodiment, the status of those patients after the censoring time may be unknown. Exemplary penalty coefficient κ may introduce a penalty term to the regression loss function for alive patients by adding a weighted absolute error between the predicted and censoring times to the loss if the predicted time of death is less than the censoring time. An exemplary penalty term may be zero if the predicted time of death is larger than or equal to the censoring time.

In an exemplary embodiment, estimating the time of death in step 108 may further include estimating a probability density function (PDF) of the time of death of intended organ donor candidate 216. FIG. 1D shows a flowchart of a method for estimating a probability density function (PDF) of a time of death of an intended organ donor candidate, consistent with one or more exemplary embodiments of the present disclosure. An exemplary method 109 may include generating a GRU-D output from intended donor clinical data 232 (step 132), generating an encoded sequence from the GRU-D output (step 134), generating a decoded sequence from the encoded sequence (step 136), generating an event-related sequence from the encoded sequence (step 138), generating a concatenated sequence by concatenating the event-related sequence and the decoded sequence (step 140), and generating the PDF of the time of death from the concatenated sequence (step 142).

FIG. 2D shows a block diagram of a sequence-to-sequence (seq2seq) neural network for time of death estimation, consistent with one or more exemplary embodiments of the present disclosure. In an exemplary embodiment, different steps of method 109 may be implemented utilizing a seq2seq neural network 207. In an exemplary embodiment, seq2seq neural network 207 may include an implementation of estimation block 206. In an exemplary embodiment, seq2seq neural network 207 may process longitudinal records of patients and impute missing values. In an exemplary embodiment, seq2seq neural network 207 may include a GRU-D layer 258, a first RNN 260, a second RNN 262, an attention mechanism 264, a concatenation layer 266, and a time distributed dense layer 268.

Referring to FIGS. 1D and 2D, in an exemplary embodiment, step 132 may include generating a GRU-D output 270 from intended donor clinical data 232 by applying intended donor clinical data 232 to GRU-D layer 258. In an exemplary embodiment, GRU-D layer 258 may allow for handling longitudinal records as well as imputing missing values of continuous covariates that may have been collected from patients.

For further detail with respect to step 134, in an exemplary embodiment, generating an encoded sequence 272 from GRU-D output 270 may include applying GRU-D output 270 to first RNN 260. An exemplary ensemble of GRU-D layer 258 and first RNN 260 may be referred to as an encoder 257 that encodes longitudinal measurements. In an exemplary embodiment, first RNN 260 may include a first plurality of RNN layers 261 for improving performance of encoder 257. In an exemplary embodiment, first RNN 260 may sequentially generate encoded sequence 272 for each step of the prediction horizon by observing values of encoded sequence 272 that are generated at previous steps. As a result, a smooth and virtually spike-free output may be generated by first RNN 260.

In further detail regarding step 136, in an exemplary embodiment, generating a decoded sequence 274 from encoded sequence 272 may include applying encoded sequence 272 to second RNN 262. In an exemplary embodiment, second RNN 262 may include a second plurality of RNN layers 263. In an exemplary embodiment, decoded sequence 274 may be associated with the time of death. An exemplary PDF of the time of death may be estimated based on decoded sequence 274, as described below in steps 138, 140, and 142. In an exemplary embodiment, each RNN layer of plurality of RNN layers 263 may generate the likelihood for each time step of decoded sequence 274 based on a previous hidden state of the RNN layer. In other words, the likelihood at a given time step may be generated based on the likelihoods of its previous time steps. As a result, generation of arbitrary values may be avoided, thereby making the decoded sequence 274 smooth and virtually spike-free.

In further detail with regards to step 138, in an exemplary embodiment, generating an event-related sequence 276 from encoded sequence 272 may include applying attention mechanism 264 to encoded sequence 272 based on decoded sequence 274. In an exemplary embodiment, attention mechanism 264 may be utilized for improving performance of seq2seq neural network 207 when a number of measurements for some patients may be high. In an exemplary embodiment, attention mechanism 264 may use the current state of second RNN 262 as an attention query. In an exemplary embodiment, event-related sequence 276 may be associated with the time of death. An exemplary PDF of the time of death may be estimated based on event-related sequence 276, as described below in steps 140 and 142.

For further detail with respect to step 140, in an exemplary embodiment, generating a concatenated sequence 278 may include applying event-related sequence 276 and decoded sequence 274 to concatenation layer 266. In an exemplary embodiment, concatenation layer 266 may concatenate event-related sequence 276 and decoded sequence 274 in concatenated sequence 278.

For further detail with regards to step 142, in an exemplary embodiment, generating a PDF 280 of the time of death from concatenated sequence 278 may include applying concatenated sequence 278 to time distributed dense layer 268. In an exemplary embodiment, time distributed dense layer 268 may generate each sample of PDF 280 at each time step from a corresponding sample of concatenated sequence 278 at that time step so that PDF 280 may show likelihood of death over a particular study time. In an exemplary embodiment, a softmax function may be applied to PDF 280 to further smooth and normalize PDF 280 in a predefined probability range, for example such as a range of (0, 1). An exemplary expected value of PDF 280 may be considered a predicted time of death for a patient.

Referring to FIGS. 1A, in an exemplary embodiment, estimating PDF 280 in step 108 may further include training seq2seq neural network 207 by minimizing a forward loss function based on the ICU dataset. An exemplary forward loss function may be defined by adding a cross-entropy classification loss term to a log-likelihood loss function—that is conventionally used in statistics and regression analysis—to improve estimation accuracy in presence of competing risks. Therefore, an exemplary forward loss function may be defined by the following:

$${L}_{forward} = {L}_{log} - ( {\sum }_{i = 1 , i ∈ {U}_{u}}^{{N}_{u}} {y}_{true}^{i} \times log {\sum }_{t = 1}^{{T}_{h}} {p}_{t}^{i} ) / {N}_{u}$$

where L_{forward} is the forward loss function, L_{log} is a log-likelihood loss term, yₜᵣᵤₑⁱ is ground truth data for in-hospital time of death of an i^{th} uncensored sample in the set of uncensored data of the i^{th} uncensored sample, pₜⁱ is predicted likelihood for in-hospital time of death the i^{th} uncensored sample at a time step t, and Tₕ is a number of time steps in PDF 280.

In an exemplary embodiment, step 110 may include obtaining the paired donor-recipient by pairing intended organ donor candidate 216 with intended recipient 220. FIG. 1E shows a flowchart of a method for pairing an intended organ donor candidate with an intended recipient, consistent with one or more exemplary embodiments of the present disclosure. An exemplary method 111 may include training seq2seq neural network 207 (step 144), extracting a donor feature set from intended donor clinical data 232 utilizing seq2seq neural network 207 (step 146), extracting each of a plurality of recipient feature sets from a respective recipient clinical data in recipient clinical dataset 218 utilizing seq2seq neural network 207 (step 148), grouping the donor feature set and a subset of the plurality of recipient feature sets (step 150), obtaining a plurality of mean squared errors (MSEs) by calculating MSEs between the donor feature set and each of the plurality of recipient feature sets in the subset (step 152), finding a smallest MSE among the plurality of MSEs (step 154), and pairing intended organ donor candidate 216 with a most similar recipient candidate of the plurality of recipient candidates to intended organ donor candidate 216 based on the smallest MSE (step 156).

Referring again to FIGS. 2A and 2D, in an exemplary embodiment, different steps of method 111 may be implemented utilizing seq2seq neural network 207. In an exemplary embodiment, seq2seq neural network 207 may include an implementation of donor-to-recipient pairing block 208.

For further detail with regards to step 144, in an exemplary embodiment, training seq2seq neural network 207 may include minimizing a reverse loss function based on the ICU dataset. An exemplary reverse loss function may be defined by adding a regularization term to forward loss function L_{forward} as follows:

$${L}_{reverse} = {L}_{forward} + \frac{λ}{M} {\sum }_{m = 1}^{M} \vert {w}_{m} \vert$$

where Lᵣₑᵥₑᵣₛₑ is the reverse loss function, A is a regularization coefficient, |wₘ| is an L₁ norm of a weight wₘ of an m^{th} training input of a plurality of training inputs in the ICU dataset, and M is a number of the plurality of training inputs. An exemplary regularization term may push weights of insignificant inputs of seq2seq neural network 207 toward zero so that a valuable subset of inputs may be utilized for estimating output of seq2seq neural network 207. In addition, exemplary regularized weights may be utilized for ranking valuable inputs by ranking the |wₘ| values from large to small ones based on the importance of each input in estimating PDF 280.

In further detail with respect to step 146, in an exemplary embodiment, extracting the donor feature set from intended donor clinical data 232 may include applying intended donor clinical data 232 to GRU-D layer 258. As a result, an exemplary donor feature set may be generated on PDF 280 as an output of seq2seq neural network 207.

In further detail regarding step 148, in an exemplary embodiment, extracting each of the plurality of recipient feature sets may include applying the respective recipient clinical data to GRU-D layer 258. As a result, each exemplary recipient feature set may be generated on PDF 280 as an output of seq2seq neural network 207.

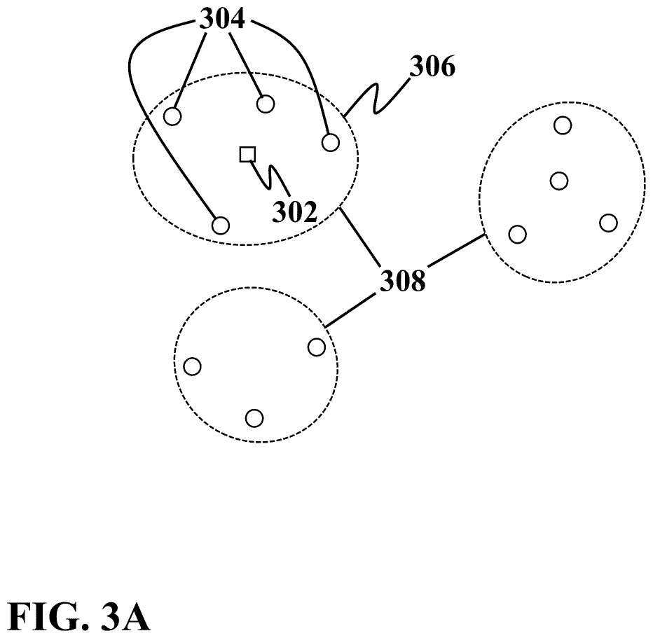

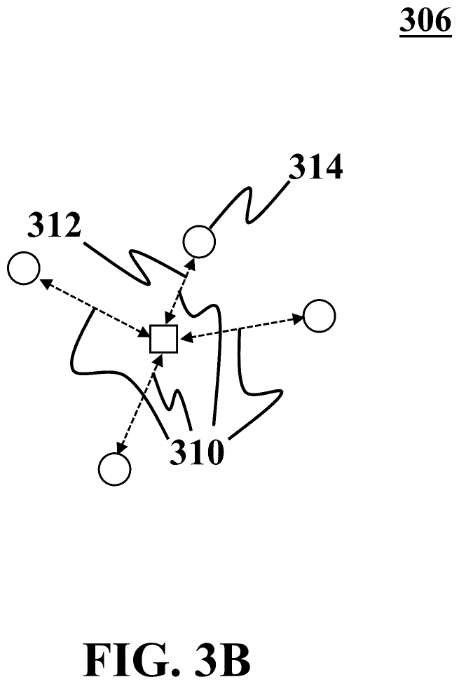

In further detail with regards to step 150, FIG. 3A shows a schematic of a plurality of clusters for grouping a donor feature set and a subset of a plurality of recipient feature sets, consistent with one or more exemplary embodiments of the present disclosure. In an exemplary embodiment, step 150 may include grouping a donor feature set 302 and a subset 304 of a plurality of recipient feature sets (represented by circular marks in FIG. 3A) in a donor cluster 306 of a plurality of clusters 308. In an exemplary embodiment, donor cluster 306 may be obtained by clustering donor feature set 302 and the plurality of recipient feature sets into plurality of clusters 308 based on distances between different feature sets among donor feature set 302 and the plurality of recipient feature sets. Since, each exemplary feature set may be a PDF, a Kolmogorov-Smirnov test may be used for measuring distances between different feature sets. In an exemplary embodiment, the Kolmogorov-Smirnov test may be used to find out a level of similarity between a pair of probability distributions. In an exemplary embodiment, a K-means clustering method may be utilized for clustering donor feature set 302 and the plurality of recipient feature sets into K disjoint groups. In an exemplary embodiment, feature sets that are grouped in donor cluster 306 may determine recipient candidates that may have transplantation outcomes similar to those of intended organ donor candidate 216 since their extracted features may have been similar to be classified in a same cluster.

In further detail with regards to step 152, FIG. 3B shows a schematic of a donor cluster, consistent with one or more exemplary embodiments of the present disclosure. Referring to FIGS. 3A and 3B, in an exemplary embodiment, step 152 may include obtaining a plurality of mean squared errors (MSEs) 310 by calculating MSEs between donor feature set 302 and each of the plurality of recipient feature sets that may be included in subset 304.

In an exemplary embodiment, step 154 may include finding a smallest MSE 312 among plurality of MSEs 310. In an exemplary embodiment, smallest MSE 312 may be associated with a most similar recipient feature set 314 (included in subset 304) to donor feature set 302. In an exemplary embodiment, a calculated MSE between donor feature set 302 and most similar recipient feature set 314 may be equal to smallest MSE 312.

In an exemplary embodiment, step 156 may include pairing intended organ donor candidate 216 with a most similar recipient candidate based on smallest MSE 312. An exemplary most similar recipient candidate may refer to a recipient candidate of whom most similar recipient feature set 314 may have been extracted. In an exemplary embodiment, if similar features are extracted from two patients, there may be a higher probability that these patients share similar features. Therefore, in an exemplary embodiment, such patients may be paired as similar patients. In an exemplary embodiment, donor-to-recipient pairing block 208 may pair two similar patients in different ways, for example, by assigning a same label (such as a number) to a pair of similar donor and recipient patients.

Referring again to FIGS. 1A, 2A, and 2B, in an exemplary embodiment, step 112 may include estimating the probability of organ transplant success for the paired donor-recipient based on intended donor clinical data 232 and intended recipient clinical data in recipient clinical dataset 218. In an exemplary embodiment, estimating the probability of organ transplant success for the paired donor-recipient may include estimating a plurality of probability density functions (PDFs) for a plurality of events for the paired donor-recipient. An exemplary plurality of PDFs may include information about probability of occurring time of each event. An exemplary plurality of events may be associated with the organ transplant success. In an exemplary embodiment, the plurality of events may include death time of intended recipient 220, a first graft failure due to early-onset pathologies (EOPs) of intended recipient 220 (such as hyperacute rejection, graft thrombosis, surgical complications, urological complications, primary non-function, and primary failure), a second graft failure due to late-onset pathologies (LOPs) of intended recipient 220 (such as infection, recurrent disease, and BK Polyoma virus), a third graft failure due to acute rejection of the intended recipient's body, a fourth graft failure due to chronic rejection of the intended recipient's body, or a fifth graft failure due to other causes.

In an exemplary embodiment, to predict the prognosis of a match, each of the plurality of PDFs may be used individually and/or collectively. Each exemplary PDF may serve as a quality index of a corresponding match. Healthcare professionals may use each exemplary PDF separately, based on the clinical situation of a candidate. In addition, by summation of normalized PDFs, a simple calculation may estimate a cumulative probability of failure over a given period of time, presenting a more comprehensive view of outcomes. In an exemplary embodiment, early failure may be defined as graft failure occurring within 12 months of transplantation, and late failure as any graft failure after that period. The information provided by each exemplary PDF may allow healthcare professionals to identify best matches based on a comprehensive insight into future events and outcomes. Even beyond transplantation, this information may be helpful in clinical decision making.

FIG. 1F shows a flowchart of a method for estimating a plurality of PDFs for a plurality of events associated with a paired donor-recipient, consistent with one or more exemplary embodiments of the present disclosure. An exemplary method 113 may include generating a latent variable from the intended donor clinical data and the intended recipient clinical data (step 157), generating a normalized output from the latent variable (step 158), generating a plurality of cause-specific outputs from the normalized output, the intended donor clinical data, and the intended recipient clinical data (step 159), generating a concatenated sequence from the plurality of cause-specific outputs (step 160), and generating each of the plurality of PDFs for each respective event of the plurality of events from the concatenated sequence (step 161).

FIG. 2E shows a block diagram of a one-to-many (one2seq) neural network, consistent with one or more exemplary embodiments of the present disclosure. In an exemplary embodiment, different steps of method 113 may be implemented utilizing a one2seq neural network 211. In an exemplary embodiment, one2seq neural network 211 may include an implementation of OMM block 210. In an exemplary embodiment, one2seq neural network 211 may include a dense network 227, a normalization layer 282, a plurality of cause-specific subnetworks 284, a concatenation layer 267, and a time distributed dense layer 269.

In an exemplary embodiment, one2seq neural network 211 may be trained by minimizing a loss function defined by adding a cross-entropy classification loss term to a conventional log-likelihood loss function, thereby improving estimation accuracy in presence of competing risks. Therefore, an exemplary loss function may be defined by the following:

$${L}_{PDF} = {L}_{log} - ( {\sum }_{e = 1}^{{N}_{e}} {\sum }_{i = 1 , i ∈ {U}_{u}}^{{N}_{u}} {y}_{true}^{e , i} \times log ( {\sum }_{t = 1}^{{T}_{h}} {p}_{t}^{e , i} ) ) / {N}_{u}$$

where L_{PDF} is the loss function, L_{log} is a log-likelihood loss term, Nₑ is a number of the plurality of events, Uᵤ is a set of uncensored data in the ICU dataset, Nᵤ is number of uncensored data in the set of uncensored data, yₜᵣᵤₑ^{e,i} is ground truth data of an i^{th} uncensored sample in the set of uncensored data for an event e of the plurality of events, pₜ^{e,i} is predicted likelihood of the i^{th} uncensored sample for event e, and Tₕ is a number of time steps in each of the plurality of PDFs. An exemplary ICU dataset may include clinical data of patients that may have been hospitalized in ICU and have a known status for each of the plurality of events. In an exemplary embodiment, yₜᵣᵤₑ^{e,i} may be set to one if event e is a first hitting event for a patient whose data is used for training one2seq neural network 211 and may be set to zero otherwise.

In an exemplary embodiment, adding the cross-entropy classification loss term to the log-likelihood loss term in loss function L_{PDF} may cause one2seq neural network 211 to predict a first hitting event (i.e., an event of the plurality of events that occurs before other events). In other words, in an exemplary embodiment, one2seq neural network 211 may generate a hazard cumulative distribution function (CDF) close to one for the first hitting event, while keeping predicted CDFs for other events close to zero, thereby increasing accuracy of estimated PDFs.

Referring to FIGS. 1F and 2E, in an exemplary embodiment, step 157 may include generating a latent variable 237 from intended donor clinical data 232 and intended recipient clinical data 233. In an exemplary embodiment, latent variable 237 may refer to a variable that is not directly observed in an output of one2seq neural network 211 but may be inferred from its output since the output may be generated from latent variable 237, as discussed later in steps 158-161.

FIG. 1G shows a flowchart for generating a latent variable from intended donor clinical data and intended recipient clinical data, consistent with one or more exemplary embodiments of the present disclosure. In an exemplary embodiment, generating latent variable 237 from intended donor clinical data 232 and intended recipient clinical data 233 may include generating a first (1^{st}) dense output of a plurality of dense outputs from intended donor clinical data 232 and intended recipient clinical data 233 (step 162), generating a first (1^{st}) dropout output of a plurality of dropout outputs from the 1^{st} dense output (step 163), generating an m^{th} dense output of the plurality of dense outputs from an (m−1)^{th} dropout output of the plurality of dropout outputs (step 164), and generating an m^{th} dropout output of the plurality of dropout outputs from the m^{th} dense output (step 165).

FIG. 2F shows a block diagram of a dense network for generating a latent variable from intended donor clinical data and intended recipient clinical data, consistent with one or more exemplary embodiments of the present disclosure. In an exemplary embodiment, different steps of flowchart 157 may be implemented utilizing dense network 227. In an exemplary embodiment, dense network 227 may include a plurality of dense layers and a plurality of dropout layers. Exemplary plurality of dense layers may include a first (1^{st}) dense layer 239 and an m^{th} dense layer 241 where 1<m≤M_{d} and M_{d} is a number of the plurality of dense layers. Neurons of each exemplary dense layer may be connected to every neuron of a preceding dense layer. Exemplary plurality of dropout layers may include a first (1^{st}) dropout layer 243 and an m^{th} dropout layer 245. In an exemplary embodiment, each dropout layer may perform a dropout process on its input. An exemplary dropout process may eliminate one or more elements of inputs of each dropout layer in a training phase of dense network 227 with a predefined probability that may be adjusted such that a negative impact of overfitting is suppressed.

Referring to FIGS. 1G and 2F, in an exemplary embodiment, step 162 may include generating a first (1^{st}) dense output 247 of the plurality of dense outputs by applying intended donor clinical data 232 and the intended recipient clinical data 233 to 1^{st} dense layer 239. In an exemplary embodiment, generating a 1^{st} dropout output 249 of the plurality of dropout outputs in step 163 may include applying 1^{st} dense output 247 to 1^{st} dropout layer 243. In an exemplary embodiment, 1^{st} dropout layer 243 may perform a dropout process on 1^{st} dense output 247.

In further detail regarding step 164, in an exemplary embodiment, generating an m^{th} dense output 251 of the plurality of dense outputs may include applying an (m−1)^{th} dropout output 253 of the plurality of dropout outputs to m^{th} dense layer 241. In an exemplary embodiment, generating an m^{th} dropout output 255 of the plurality of dropout outputs in step 165 may include applying m^{th} dense output 251 to m^{th} dropout layer 245. In an exemplary embodiment, m^{th} dropout layer 245 may perform a dropout process on m^{th} dense output 251. An exemplary M_{d}^{th} dropout output of the plurality of dropout outputs may include latent variable 237.

Referring again to FIGS. 1F and 2E, in an exemplary embodiment, step 158 may include generating a normalized output 286 from latent variable 237 by applying latent variable 237 to normalization layer 282. In an exemplary embodiment, normalization layer 282 may perform a batch normalization process on latent variable 237. In an exemplary embodiment, the batch normalization process may normalize latent variable 237 utilizing an average and a standard deviation of a set of the latent variable samples that are associated with a batch of training data. In doing so, training data may be partitioned into batches. Next, an exemplary sct of the latent variable samples may be obtained from the batch. Afterwards, in an exemplary embodiments, an average and a standard deviation of the set of latent variable samples may be obtained and all elements of the set may be normalized in accordance to the average and the standard deviation. Next, in an exemplary embodiments, all elements of the set may be scaled and shifted by a scale and a shift variable which may be learned during a training process. Therefore, in an exemplary embodiments, all elements of latent variable 237 may follow a normal distribution which may considerably reduce a required time for training one2seq neural network 211.

For further detail regarding step 159, in an exemplary embodiment, generating each of a plurality of cause-specific outputs 288 may include applying normalized output 286, intended donor clinical data 232, and intended recipient clinical data 233 to each of plurality of cause-specific subnetworks 284. In an exemplary embodiment, each of plurality of cause-specific subnetworks 284 may include a respective plurality of gated recurrent unit (GRU) layers. For example, cause-specific subnetwork 284A may include a plurality of GRU layers 285. In an exemplary embodiment, each GRU layer of plurality of GRU layers 285 may generate the likelihood for each time step of a cause-specific output 288A based on a previous hidden state of the GRU layer. In other words, the likelihood at a given time step may be generated based on the likelihoods of its previous time steps. As a result, generation of arbitrary values may be avoided, thereby making cause-specific output 288A and consequently, the estimated PDFs smooth and virtually spike-free. In addition, utilizing GRU layers in plurality of cause-specific subnetworks 284 may prevent an overfitting issue by significantly reducing the number of parameters of one2seq neural network 211.

For further detail with respect to step 160, in an exemplary embodiment, generating a concatenated sequence 279 may include applying plurality of cause-specific outputs 288 to concatenation layer 267. In an exemplary embodiment, concatenation layer 267 may concatenate plurality of cause-specific outputs 288 in concatenated sequence 279.

For further detail with regards to step 161, in an exemplary embodiment, generating each of a plurality of PDFs 281 may include applying concatenated sequence 279 to time distributed dense layer 269. In an exemplary embodiment, time distributed dense layer 269 may generate each PDF sample of plurality of PDFs 281 at each time step from a corresponding sample of concatenated sequence 279 at that time step so that each PDF of plurality of PDFs 281 may show likelihood of a corresponding event. In an exemplary embodiment, a softmax function may be applied to each of a plurality of PDFs 281 to further smooth and normalize each PDF in a predefined probability range, for example such as a range of (0, 1).

Referring again to FIG. 1A, in an exemplary embodiment, step 114 may include pairing intended recipient 220 with the plurality of intended organ donors. FIG. 1H shows a flowchart for pairing an intended recipient with a plurality of intended organ donors, consistent with one or more exemplary embodiments of the present disclosure. In an exemplary embodiment, pairing intended recipient 220 with the plurality of intended organ donors may include training a sequence-to-sequence (seq2seq) neural network based on the ICU dataset (step 166), extracting a recipient feature set from the intended recipient clinical data 233 utilizing the seq2seq neural network (step 168), extracting each of a plurality of donor feature sets from a respective donor clinical data in donor clinical dataset 214 utilizing the seq2seq neural network by applying the respective donor clinical data to the seq2seq neural network (step 170), grouping the recipient feature set and a subset of the plurality of donor feature sets (step 172), obtaining a plurality of mean squared errors (MSEs) by calculating MSEs between the recipient feature set and each of the plurality of donor feature sets in the subset (step 174), extracting an MSE subset from the plurality of MSEs (step 176), extracting an organ donor candidates subset from the plurality of organ donor candidates (step 178), and pairing intended recipient 220 with each organ donor candidate in the organ donor candidates subset (step 180).

FIG. 2G shows a block diagram of a sequence-to-sequence (seq2seq) neural network for pairing an intended recipient with a plurality of intended organ donors, consistent with one or more exemplary embodiments of the present disclosure. In an exemplary embodiment, different steps of flowchart 114 may be implemented utilizing a seq2seq neural network 213. In an exemplary embodiment, seq2seq neural network 213 may include an implementation of recipient-to-donor pairing block 212. In an exemplary embodiment, seq2seq neural network 213 may be used for post-graft predictions, as seq2seq neural network 213 may be able to handle longitudinal post-graft data. In an exemplary embodiment, seq2seq neural network 213 may include a GRU-D layer 259, an encoder RNN 290, a plurality of decoder RNNs (for example, decoder RNNs 292A and 292B), an attention mechanism 265, a plurality of concatenation layers 294, and a plurality of time distributed dense layers 296.

For further detail with regards to step 166, in an exemplary embodiment, training seq2seq neural network 213 may include minimizing a reverse loss function based on the ICU dataset. An exemplary reverse loss function may be defined similar to loss function Lᵣₑᵥₑᵣₛₑ described above in step 144. An exemplary ICU dataset may include clinical data of patients that may have been hospitalized in ICU and have a known status for each of a plurality of events that are associated with each patient, as described below.

In an exemplary embodiment, step 168 may include extracting the recipient feature set from intended recipient clinical data 233 by applying intended recipient clinical data 233 to seq2seq neural network 207. In an exemplary embodiment, step 170 may include extracting each of the plurality of donor feature sets from a respective donor clinical data that may be stored in donor clinical dataset 214 by applying the respective donor clinical data to seq2seq neural network 207. In other words, each exemplary donor feature set may be extracted from a separate donor clinical data in donor clinical dataset 214.

In further detail regarding steps 168 and 170, in an exemplary embodiment, applying intended recipient clinical data 233 to seq2seq neural network 213 or applying a donor clinical data to seq2seq neural network 213 may include estimating a plurality of probability density functions (PDFs) for a plurality of events from input data. An exemplary input data may include intended recipient clinical data 233 or a donor clinical data. An exemplary plurality of events may be associated with intended recipient 220 or an organ donor candidate of the plurality of organ donor candidates. In an exemplary embodiment, the plurality of events may include death time of a patient (i.e., intended recipient 220 or an organ donor candidate), a first graft failure due to early-onset pathologies (EOPs) of a patient (such as hyperacute rejection, graft thrombosis, surgical complications, urological complications, primary non-function, and primary failure), a second graft failure due to late-onset pathologies (LOPs) of a patient (such as infection, recurrent disease, and BK Polyoma virus), a third graft failure due to acute rejection of a patient's body, a fourth graft failure due to chronic rejection of a patient's body, or a fifth graft failure due to other causes.

FIG. 1I shows a flowchart of a method for estimating a plurality of PDFs for a plurality of events associated with an intended recipient or an organ donor candidate, consistent with one or more exemplary embodiments of the present disclosure. Referring to FIGS. 11 and 2G, an exemplary method 169 may include generating a gated recurrent unit with trainable decays (GRU-D) output 271 from input data 298 (step 182), generating an encoded sequence 273 from GRU-D output 271 (step 184), generating a plurality of decoded sequences (for example, decoded sequences 275A and 275B) from encoded sequence 273 (step 186), generating a plurality of event-related sequences (for example, event-related sequences 277A and 277B) from encoded sequence 273 based on a respective decoded sequence of the plurality of decoded sequences (step 188), generating a plurality of concatenated sequences (for example, concatenated sequences 278A and 278B) by concatenating each of the plurality of event-related sequences and a respective decoded sequence of the plurality of decoded sequences (step 190), and generating each of a plurality of PDFs 299 for each respective event of the plurality of events from a respective concatenated sequence of the plurality of concatenated sequences (step 192).

In further detail regarding step 182, in an exemplary embodiment, generating GRU-D output 271 may include applying input data 298 to GRU-D layer 259. In an exemplary embodiment, GRU-D layer 259 may allow for handling longitudinal records as well as imputing missing values of continuous covariates that may have been collected from patients.

For further detail with respect to step 184, in an exemplary embodiment, generating encoded sequence 273 may include applying GRU-D output 271 to encoder RNN 290. In an exemplary embodiment, encoder RNN 290 may include a first plurality of RNN layers 291. In an exemplary embodiment, each RNN layer of first plurality of RNN layers 291 may generate the likelihood for each time step of encoded sequence 273 based on a previous hidden state of the RNN layer. In other words, the likelihood at a given time step may be generated based on the likelihoods of its previous time steps. As a result, generation of arbitrary values may be avoided, thereby making the encoded sequence 273 smooth and virtually spike-free.

For further detail with regards to step 186, in an exemplary embodiment, generating the plurality of decoded sequences may include applying encoded sequence 273 to the plurality of decoder RNNs. For example, decoded sequence 275A may be obtained by applying encoded sequence 273 to decoder RNN 292A and decoded sequence 275B may be obtained by applying encoded sequence 273 to decoder RNN 292B. In an exemplary embodiment, each of the plurality of decoder RNNs may include a respective second plurality of RNN layers. For example, decoder RNN 292A may include a second plurality of RNN layers 293A and decoder RNN 292B may include a second plurality of RNN layers 293B. In an exemplary embodiment, each RNN layer of second plurality of RNN layers 293A may generate the likelihood for each time step of decoded sequence 275A based on a previous hidden state of the RNN layer. In other words, the likelihood at a given time step may be generated based on the likelihoods of its previous time steps. As a result, generation of arbitrary values may be avoided, thereby making the decoded sequence 275A smooth and virtually spike-free.

In further detail with respect to step 188, in an exemplary embodiment, generating each the plurality of event-related sequences may include applying attention mechanism 265 to encoded sequence 273 based on a respective decoded sequence of the plurality of decoded sequences. For example, event-related sequence 277A may be obtained by applying attention mechanism 265 to encoded sequence 273 based on decoded sequence 275A and event-related sequence 277B may be obtained by applying attention mechanism 265 to encoded sequence 273 based on decoded sequence 275B. In an exemplary embodiment, attention mechanism 265 may be utilized for improving performance of seq2seq neural network 213 when a number of measurements for some patients may be high. In an exemplary embodiment, attention mechanism 265 may use the current state of each of the plurality of decoder RNNs as a respective attention query. For example, the current state of decoder RNN 292A may be utilized by attention mechanism 265 as an attention query for generating event-related sequence 277A.

In further detail regarding step 190, in an exemplary embodiment, generating each of the plurality of concatenated sequences (for example, concatenated sequences 278A and 278B) may include applying each respective event-related sequence and respective decoded sequence to a respective concatenation layer of plurality of concatenation layers 294. For example, concatenated sequence 278A may be obtained by applying event-related sequence 277A and decoded sequence 275A to concatenation layer 294A and concatenated sequence 278B may be obtained by applying event-related sequence 277B and decoded sequence 275B to concatenation layer 294B. In an exemplary embodiment, each of plurality of concatenation layers 294 may concatenate a respective event-related sequence and a respective decoded sequence. For example, concatenation layer 294A may concatenate vent-related sequence 277A and decoded sequence 275A in concatenated sequence 278A and concatenation layer 294B may concatenate vent-related sequence 277B and decoded sequence 275B in concatenated sequence 278B.

For further detail with regards to step 192, in an exemplary embodiment, generating each of plurality of PDFs 299 may include applying each respective concatenated sequence to a respective time distributed dense layer. For example, a PDF 299A may be obtained by applying concatenated sequence 278A to a time distributed dense layer 296A and a PDF 299B may be obtained by applying concatenated sequence 278B to a time distributed dense layer 296B. In an exemplary embodiment, time distributed dense layer 296A may generate each sample of PDF 299A at each time step from a corresponding sample of concatenated sequence 278A at that time step so that PDF 299A may show likelihood of a corresponding event. In an exemplary embodiment, a softmax function may be applied to each of a plurality of PDFs 299 to further smooth and normalize each PDF in a predefined probability range, for example such as a range of (0, 1).

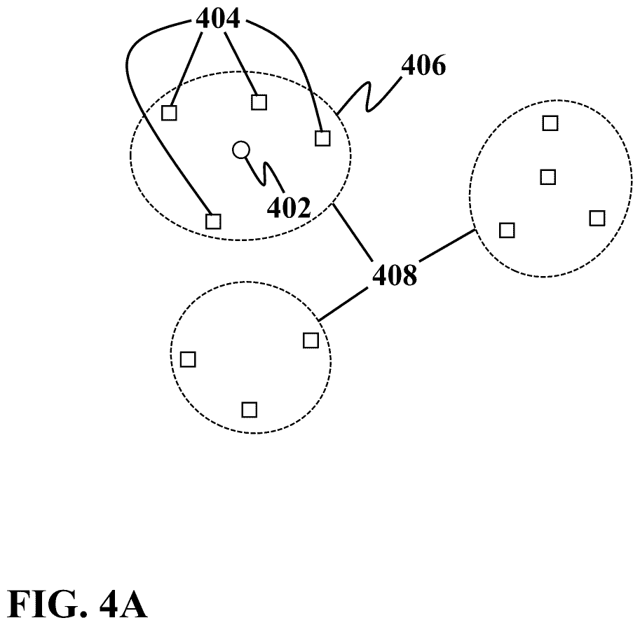

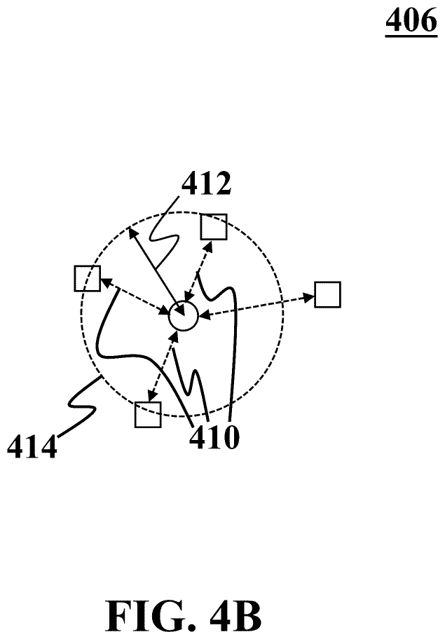

Referring again to FIG. 1H, in an exemplary embodiment, step 172 may include grouping the recipient feature set and the subset of the plurality of donor feature sets in a recipient cluster of a plurality of clusters. FIG. 4A shows a schematic of a plurality of clusters for grouping a recipient feature set and a subset of a plurality of donor feature sets, consistent with one or more exemplary embodiments of the present disclosure. In an exemplary embodiment, a recipient feature set 402 and a subset 404 of a plurality of donor feature sets (represented by square marks in FIG. 4A) may be grouped in a recipient cluster 406 of a plurality of clusters 408. In an exemplary embodiment, recipient cluster 406 may be obtained by clustering recipient feature set 402 and the plurality of donor feature sets into plurality of clusters 408 based on distances between different feature sets among recipient feature set 402 and the plurality of donor feature sets. As discussed above in steps 168 and 170, each exemplary feature set may include a plurality of PDFs. Therefore, in an exemplary embodiment, a Jensen-Shannon divergence method may be used for measuring distances between different feature sets. In an exemplary embodiment, the Jensen-Shannon divergence method may be used to find out a level of similarity between different probability distributions in a symmetric way. In an exemplary embodiment, a K-means clustering method may be utilized for clustering recipient feature set 402 and the plurality of donor feature sets into K disjoint groups.

In further detail with regards to step 174, FIG. 4B shows a schematic of a recipient cluster, consistent with one or more exemplary embodiments of the present disclosure. Referring to FIGS. 4A and 4B, in an exemplary embodiment, step 174 may include obtaining a plurality of MSEs (represented by dashed arrows in FIG. 4B) by calculating MSEs between recipient feature set 402 and each of the plurality of donor feature sets that may be included in subset 404.

In further detail with regards to step 176, in an exemplary embodiment, extracting an MSE subset 410 may include extracting MSEs from the plurality of MSEs that may have values smaller than an MSE threshold 412. Exemplary MSEs in MSE subset 410 may be located inside a circle 414 with a radius equal to MSE threshold 412.

In further detail with regards to step 178, each exemplary organ donor candidate in the organ donor candidates subset may be associated with a respective MSE in MSE subset 410. Therefore, an organ donor candidates subset may be extracted by selecting each organ donor candidate whose extracted feature set (i.e., a feature set that has been extracted from clinical data acquired from the organ donor candidate as described above in step 170) is closer to recipient feature set 402 than MSE threshold 412 in terms of MSE (i.e., a calculated MSE for the feature set of the organ donor candidate is smaller than MSE threshold 412).

In an exemplary embodiment, step 180 may include pairing intended recipient 220 with each organ donor candidate in the organ donor candidates subset. In an exemplary embodiment, if similar features are extracted from different patients, there may be a higher probability that these patients share similar features. Therefore, in an exemplary embodiment, such patients may be paired as similar patients. In an exemplary embodiment, recipient-to-donor pairing block 212 may pair intended recipient 220 with patients in the organ donor candidates subset in different ways, for example, by assigning a same label (such as a number) to a group of similar recipient and donor patients.

Referring again to FIGS. 2A-2G, in an exemplary embodiment, GRU-D neural network 205, seq2seq neural network 207, one2seq neural network 211, and seq2seq neural network 213 may include Bayesian neural networks (BNNs). In an exemplary embodiment, instead of a single value, a random variable with a Gaussian distribution may be assigned to each weight of a BNN. Exemplary mean and standard deviation of each Gaussian distribution may be estimated for each weight. As a result, exemplary BNNs may be able to predict multiple PDFs per prediction. In addition, exemplary BNNs may allow for describing possible randomness and uncertainty in trained weights of different networks in system 200 as well as uncertainty of predictions. As a result, exemplary predictions may become interpretable which may show a level of confidence in different predictions. An exemplary prediction may be reliable when it has high confidence. On the other hand, low confidence for an exemplary BNN's prediction may imply that the prediction is not reliable. Exemplary BDNNs may also be able to address overfitting problems by taking advantage of Bayesian learning and incorporating a prior distribution for each weight of a neural network in system 200.

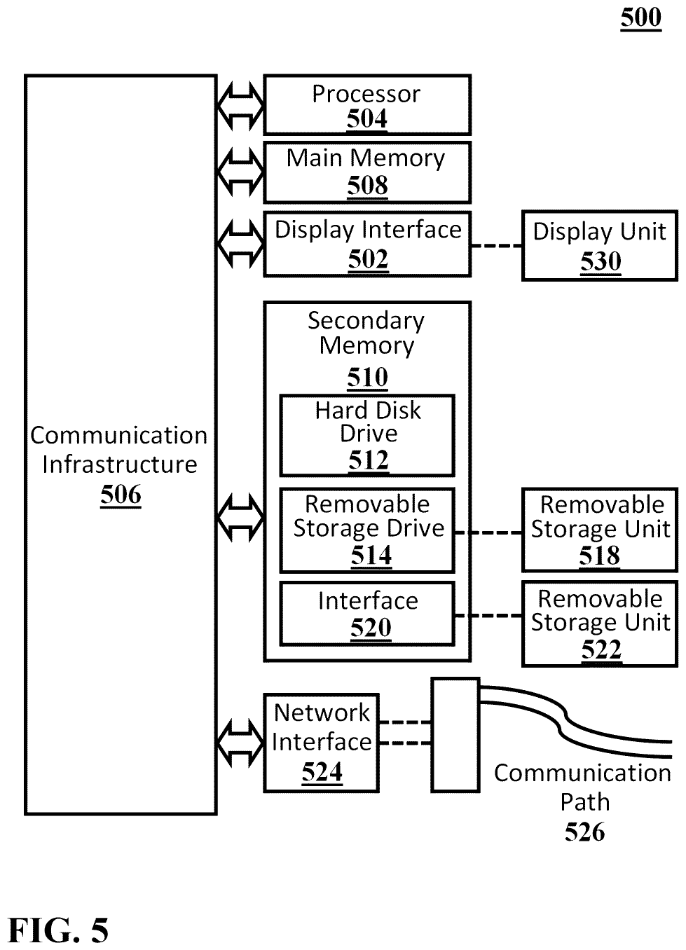

FIG. 5 shows an example computer system 500 in which an embodiment of the present invention, or portions thereof, may be implemented as computer-readable code, consistent with exemplary embodiments of the present disclosure. For example, different steps of method 100 may be implemented in computer system 500 using hardware, software, firmware, tangible computer readable media having instructions stored thereon, or a combination thereof and may be implemented in one or more computer systems or other processing systems. Hardware, software, or any combination of such may embody any of the modules and components in FIGS. 1A-4B.

If programmable logic is used, such logic may execute on a commercially available processing platform or a special purpose device. One ordinary skill in the art may appreciate that an embodiment of the disclosed subject matter can be practiced with various computer system configurations, including multi-core multiprocessor systems, minicomputers, mainframe computers, computers linked or clustered with distributed functions, as well as pervasive or miniature computers that may be embedded into virtually any device.

For instance, a computing device having at least one processor device and a memory may be used to implement the above-described embodiments. A processor device may be a single processor, a plurality of processors, or combinations thereof. Processor devices may have one or more processor “cores.”

An embodiment of the invention is described in terms of this example computer system 300. After reading this description, it will become apparent to a person skilled in the relevant art how to implement the invention using other computer systems and/or computer architectures. Although operations may be described as a sequential process, some of the operations may in fact be performed in parallel, concurrently, and/or in a distributed environment, and with program code stored locally or remotely for access by single or multiprocessor machines. In addition, in some embodiments the order of operations may be rearranged without departing from the spirit of the disclosed subject matter.

Processor device 504 may be a special purpose (e.g., a graphical processing unit) or a general-purpose processor device. As will be appreciated by persons skilled in the relevant art, processor device 504 may also be a single processor in a multi-core/multiprocessor system, such system operating alone, or in a cluster of computing devices operating in a cluster or server farm. Processor device 504 may be connected to a communication infrastructure 506, for example, a bus, message queue, network, or multi-core message-passing scheme.

In an exemplary embodiment, computer system 500 may include a display interface 502, for example a video connector, to transfer data to a display unit 530, for example, a monitor. Computer system 500 may also include a main memory 508, for example, random access memory (RAM), and may also include a secondary memory 510. Secondary memory 510 may include, for example, a hard disk drive 512, and a removable storage drive 514. Removable storage drive 514 may include a floppy disk drive, a magnetic tape drive, an optical disk drive, a flash memory, or the like. Removable storage drive 514 may read from and/or write to a removable storage unit 518 in a well-known manner. Removable storage unit 518 may include a floppy disk, a magnetic tape, an optical disk, etc., which may be read by and written to by removable storage drive 514. As will be appreciated by persons skilled in the relevant art, removable storage unit 518 may include a computer usable storage medium having stored therein computer software and/or data.

In alternative implementations, secondary memory 510 may include other similar means for allowing computer programs or other instructions to be loaded into computer system 500. Such means may include, for example, a removable storage unit 522 and an interface 520. Examples of such means may include a program cartridge and cartridge interface (such as that found in video game devices), a removable memory chip (such as an EPROM, or PROM) and associated socket, and other removable storage units 522 and interfaces 520 which allow software and data to be transferred from removable storage unit 522 to computer system 500.

Computer system 500 may also include a communications interface 524. Communications interface 524 allows software and data to be transferred between computer system 500 and external devices. Communications interface 524 may include a modem, a network interface (such as an Ethernet card), a communications port, a PCMCIA slot and card, or the like. Software and data transferred via communications interface 524 may be in the form of signals, which may be electronic, electromagnetic, optical, or other signals capable of being received by communications interface 524. These signals may be provided to communications interface 524 via a communications path 526. Communications path 526 carries signals and may be implemented using wire or cable, fiber optics, a phone line, a cellular phone link, an RF link or other communications channels.

In this document, the terms “computer program medium” and “computer usable medium” are used to generally refer to media such as removable storage unit 518, removable storage unit 522, and a hard disk installed in hard disk drive 512. Computer program medium and computer usable medium may also refer to memories, such as main memory 508 and secondary memory 510, which may be memory semiconductors (e.g. DRAMs, etc.).

Computer programs (also called computer control logic) are stored in main memory 508 and/or secondary memory 510. Computer programs may also be received via communications interface 524. Such computer programs, when executed, enable computer system 500 to implement different embodiments of the present disclosure as discussed herein. In particular, the computer programs, when executed, enable processor device 504 to implement the processes of the present disclosure, such as the operations in method 100 illustrated by flowcharts of FIGS. 1A-FIG. 1I discussed above. Accordingly, such computer programs represent controllers of computer system 500. Where an exemplary embodiment of method 100 is implemented using software, the software may be stored in a computer program product and loaded into computer system 500 using removable storage drive 514, interface 320, and hard disk drive 512, or communications interface 524.

Embodiments of the present disclosure also may be directed to computer program products including software stored on any computer useable medium. Such software, when executed in one or more data processing device, causes a data processing device to operate as described herein. An embodiment of the present disclosure may employ any computer useable or readable medium. Examples of computer useable mediums include, but are not limited to, primary storage devices (e.g., any type of random access memory), secondary storage devices (e.g., hard drives, floppy disks, CD ROMS, ZIP disks, tapes, magnetic storage devices, and optical storage devices, MEMS, nanotechnological storage device, etc.).

The embodiments have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

### Example

In this example, performance of an implementation of method 100 for identifying a plurality of intended organ donors is demonstrated. Different steps of the method are implemented utilizing an implementation of system 200. In order to train modules for time of death prediction (for example, an implementation DCD module 209), the medical information mart for intensive care-III (MIMIC-III) dataset disclosed by Johnson et al. in “MIMIC-III, a freely accessible critical care database.” Scientific data 3, no. 1 (2016): 1-9, is used. The database contains data of 53,423 distinct admitted ICU adults (16 years old or above) between 2001 and 2012. The dataset includes several observations over time per patient, i.e., longitudinal data during the ICU stay including vital signs, administered fluids, laboratory measurements, microbiology information, excreted fluids, and prescriptions. Out of 16085 covariates, a list of 1072 are identified potentially relevant covariates that are commonly measured in ICUs. The selected covariates of each patient's ID are combined to obtain the whole set of recorded data for that patient during the ICU admission. The MIMIC-III dataset is cleaned up, addressing anomalies and errors using state-of-the-art data analysis techniques. Among different causes of death, only “circulatory deaths”, defined as irreversible loss of function of the heart and lung, is included. Patients who died within 28 days after admission are included in training, as this time period is deemed enough for the purpose of preparing a potential donor.

The scientific registry of transplant recipients (SRTR) dataset disclosed by Kim et al. in “OPTN/SRTR 2016 annual data report: liver.” American Journal of Transplantation 18 (2018): 172-253, is used for training implementations of OMM block 210 and recipient-to-donor pairing block 212 for matchmaking and transplant monitoring. However, SRTR contains only 10% of the total measurements. Out of 1093 covariates, a list of 472 potentially relevant covariates (pre-graft and postgraft) that are commonly measured in healthcare systems are identified. They are then combined based on IDs of each donor-recipient pair to obtain the full record of the transplants. The SRTR dataset contains records of about 480,000 pre-graft (paired donors and recipients) and 460,000 post-graft (recipient's follow-up data) kidney transplants. According to SRTR, graft failure is defined as irreversible loss of function of a grafted kidney, re-transplanted or not.

A combination of non-longitudinal pre-graft data and longitudinal postgraft data is prepared to train implementations of one2seq neural network 211 and seq2seq neural network 213 that are utilized for predicting hazard rates for death and graft failure at any time point, from matchmaking to a time when cither a graft fails or a patient dies. Patients with a death or graft failure event within 20 years after transplantation are included in training. MIMIC-III and SRTR datasets are split into 80% training and 20% testing sets.

Different metrics are used for evaluating core performance of implementations of method 100 and system 200, including mean absolute error (MAE) that is an absolute difference between an expected value of an estimated PDF and the ground truth (lower values indicate higher accuracy), F1 score which is a value in the range [0, 1] and is used for measuring classification accuracy (higher scores indicate better accuracy), area under the ROC curve (AUC) which is in the range [0, 1] and is used for measuring classification accuracy (higher scores indicate better accuracy), and time horizon (TH) which is a period of time for which the performance of a model is evaluated. Time horizons are cumulative, not disjoint. This means that each TH contains all patients for all previous THs. The cumulativeness of time horizons is necessary to avoid any bias of a system towards certain parts of data distribution. To evaluate an implementation of DCD module 209, accumulative THs are defined as TH1=72 hours (3 days), TH2=168 hours (1 week), TH3=504 hours (3 weeks), and TH4=672 hours (4 weeks). As an example, patients that are predicted to die within three days of admission to ICU are categorized in TH1. To evaluate an implementation of OMM block 210 and recipient-to-donor pairing block 212, accumulative THs are defined as TH1=12 months, TH2-60 months, TH3=120 months, and TH4=240 months. As an example, patients that are predicted to die or have graft failure within 12 months of transplantation are categorized in the TH1.

Since, measurements in healthcare are performed incrementally over time, the performance of implemented systems and methods is evaluated in a simulated environment in which data is supplied incrementally to mimic the real world conditions. Therefore, an incremental mean absolute error (IMAE) is defined as an error measure when each prediction is calculated based on sequential observations over time. For example, IMAE for ICU patients shows the average error expected for predicting death time over each time horizon. Therefore, a core performance result is expected to have better accuracy compared to simulation performance since all sequential observations are already available when calculating the core performance. IMAE is used to evaluate the accuracy of organ failure predictions at each observation sequence.

Core performance accuracy of implementations of GRU-D neural network 205 and seq2seq neural network 207 in DCD module 209 are presented in Tables 1 and 2, respectively. The range for F1 score and AUC is [0, 1] with values closer to 1 indicating a better performance. Therefore, the scores >0.9 in Table 1 for all time horizons (TH) show that GRU-D neural network 205 can effectively predict the event of death for ICU patients. Generally, more longitudinal measurements are recorded for patients for whom primary events occur later, leading to more accurate predictions as TH widens. Hence, an overall performance of an implementation of prediction block 204 increases in longer THs.

Referring to Table 2, MAE values for an implementation of seq2seq neural network 207 increase for longer THs. As TH widens, patients with longer survival times are added to the test set. The absolute prediction error for such patients is larger than that for patients with shorter survival times. A lower MAE indicates higher accuracy. For each MAE in Table 2, a confidence interval (including a lower bound and an upper bound of the estimated MAE) at a 95% confidence level is also provided.

TABLE 1
Core performance of an
implementation of GRU-D neural network 205.

| Accuracy Metrics/Time Horizon | TH1 | TH2 | TH3 | TH4 |
|---|---|---|---|---|
| F1 Score | 0.92 | 0.93 | 0.94 | 0.94 |
| AUC | 0.90 | 0.90 | 0.92 | 0.91 |

TABLE 2
Core performance of an
implementation of seq2seq neural network 207.

| Time Horizon | TH1 | TH2 | TH3 | TH4 |
|---|---|---|---|---|
| MAE | 17 | 21 | 30 | 34 |
| | (13.1, 20.9) | (17.1, 24.9) | (25.9, 34.1) | (28.5, 39.5) |

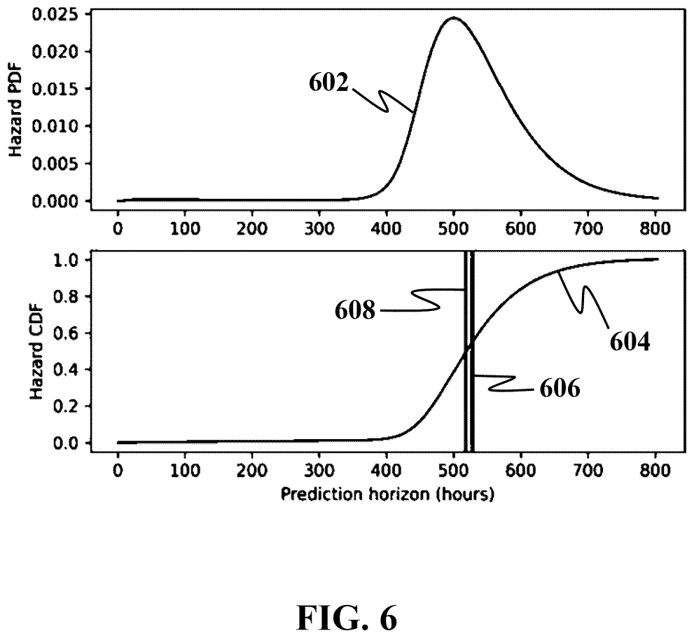

FIG. 6 shows a PDF 602, a CDF 604, and a predicted time of death 606 predicted by a seq2seq neural network, consistent with one or more exemplary embodiments of the present disclosure. Besides the closeness of predicted time of death 606 to a ground truth 608, the smoothness of generated PDF 602 is exceptional.

To study the applicability of an implementation of DCD module 209 to real-life practice, Table 3 shows results of an implementation of GRU-D neural network 205 in a simulated environment. According to Table 3, an implementation of GRU-D neural network 205 is highly accurate in predicting death occurrences. Considering AUC values in Table 3, an implementation of GRU-D neural network 205 generates more false positive predictions for patients who are discharged from ICU within 72 hours (TH1), an expected phenomenon as described above.

TABLE 3
Results of an implementation of GRU-D neural
network 205 in a simulated environment.

| Accuracy Metrics/Time Horizon | TH1 | TH2 | TH3 | TH4 |
|---|---|---|---|---|
| F1 Score | 0.92 | 0.94 | 0.94 | 0.94 |
| AUC | 0.76 | 0.84 | 0.88 | 0.89 |

Table 4 shows results of an implementation of seq2seq neural network 207 in a simulated environment based on the IMAE metrics. For each IMAE in Table 4, a confidence interval (including a lower bound and an upper bound of the estimated IMAE) at a 95% confidence level is also provided. It may be expected that seq2seq neural network 207 may have an average error of about 19 hours in predicting time of death for patients staying in ICU for less than 72 hours (TH1). Prediction of death time in advance may provide health systems with a valuable time to assess suitability of patients for donation and start executive processes.

TABLE 4
Results of an implementation of seq2seq neural
network 207 in a simulated environment.

| Time Horizon | TH1 | TH2 | TH3 | TH4 |
|---|---|---|---|---|
| IMAE | 19 | 33 | 49 | 59 |
| | (16.4, 21.6) | (28.5, 37.5) | (41.1, 56.9) | (48.5, 69.5) |

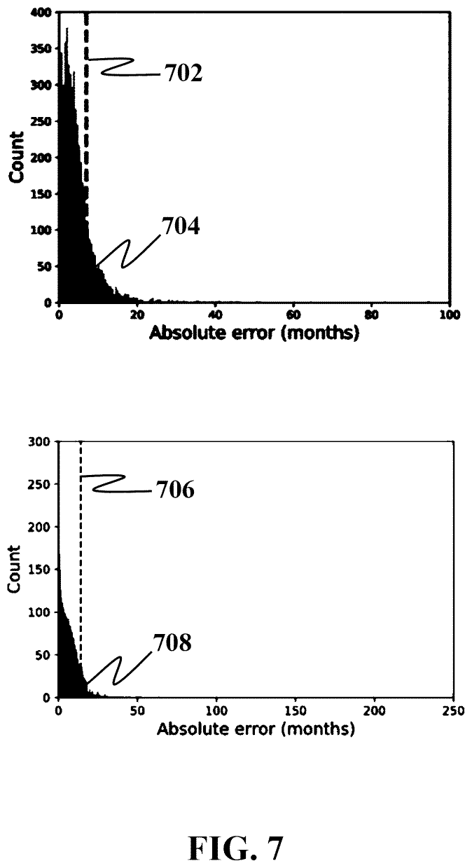

Outcomes predicted by implementations of OMM block 210 and recipient-to-donor pairing block 212 include probability and time of a recipient's death (non-traumatic, non-suicidal), as well as the probability and time of graft failure categorized by underlying pathology. Tables 5 and 6 show the accuracy performances of implementations of one2seq neural network 211 and seq2seq neural network 213, respectively. For each MAE in Tables 5 and 6, a confidence interval at a 95% confidence level is also provided. FIG. 7 shows error distribution of a one2seq neural network and a seq2seq neural network, consistent with one or more exemplary embodiments of the present disclosure. Standard deviations of error are indicated by dotted lines.

TABLE 5
Core performance of an implementation of one2seq
neural network 211 using MAE in months.

| Outcomes | TH1 | TH2 | TH3 | TH4 |
|---|---|---|---|---|
| Death | 2.4 (2.08, 2.72) | 6.6 (6.16, 7.04) | 6.5 (6.15, 6.85) | 7.3 (6.92, 7.68) |
| GF-AC | 1.9 (1.78, 2.02) | 5.6 (5.42, 5.79) | 6.1 (5.93, 6.26) | 6.7 (6.62, 6.97) |
| GF-EOP | 1.3 (1.14, 1.46) | 2.9 (2.6, 3.2) | 3.6 (3.33, 3.93) | 4.7 (4.28, 5.12) |
| GF-LOP | 3.0 (2.66, 3.34) | 7.1 (6.69, 7.51) | 7.4 (7.03, 7.77) | 7.9 (7.52, 8.28) |
| GF-AR | 2.3 (2.06, 2.54) | 5.8 (5.45, 6.15) | 6.3 (5.97, 6.33) | 6.7 (6.35, 7.05) |
| GF-CR | 3.4 (2.66, 4.14) | 7.6 (7.13, 8.07) | 7.0 (6.65, 7.35) | 7.3 (6.95, 7.65) |
| GF-OC | 1.8 (1.52, 2.08) | 6.2 (5.71, 6.69) | 6.5 (6.12, 6.88) | 7.5 (7.08, 7.92) |

TABLE 6
Core performance of an implementation of seq2seq
neural network 213 using MAE in months.

| Outcomes | TH1 | TH2 | TH3 | TH4 |
|---|---|---|---|---|
| Death | 5.2 (4.19, 6.21) | 5.8 (5.14, 6.46) | 6.2 (5.66, 6.74) | 6.4 (5.84, 6.96) |
| GF-AC | 4.5 (4.21, 4.79) | 4.3 (4.11, 4.49) | 4.5 (4.34, 4.66) | 5.1 (4.90, 5.51) |
| GF-EOP | 4.5 (4.17, 4.83) | 4.6 (4.41, 4.79) | 4.8 (4.64, 4.96) | 5.3 (5.09, 5.51) |
| GF-LOP | 4.3 (3.62, 4.98) | 4.5 (3.84, 5.16) | 4.5 (4.00, 5.00) | 5.6 (4.76, 6.44) |
| GF-AR | 4.1 (3.84, 4.72) | 3.9 (3.55, 4.25) | 4.1 (3.79, 4.47) | 4.8 (4.31, 5.29) |
| GF-CR | 5.0 (4.36, 5.64) | 4.2 (3.90, 4.50) | 4.2 (3.93, 4.47) | 4.6 (4.29, 4.91) |
| GF-OC | 3.6 (2.55, 4.65) | 4.9 (4.37, 5.43) | 4.9 (4.51, 5.29) | 5.6 (5.09, 6.11) |

Comparing core performances of an implementation of one2seq neural network 211 (Table 5) and seq2seq neural network 213 (Table 6) reveals that the accuracy of predictions of an implementation of seq2seq neural network 213 increases for late onset pathologies and decreases for early onset pathologies. The lower accuracy of an implementation of seq2seq neural network 213 in predicting early onset pathologies is mainly due to the low frequency of measurements in SRTR (only once in the 1^{st} year), too few for a recurrent model for accurate predictions early after transplantation. Of note, a standard deviation 702 for an error distribution 704 of an implementation of seq2seq neural network 213 is smaller than a standard deviation 706 for an error distribution 708 of an implementation of one2seq neural network 211.

In the practice of transplantation, matchmaking is performed in two stages, including clinical matchmaking and cross-matching for those predicted to be good matches. Matchmaking is performed twice using an implementation of one2seq neural network 211, once with pre-graft data excluding crossmatch results, and once including them. Table 7 shows the performance of an implementation of one2seq neural network 211 after crossmatch. For each MAE in Table 7, a confidence interval at a 95% confidence level is also provided. As expected, Tables 7 shows that MAE for an implementation of one2seq neural network 211 decreases only by an average of about 0.9 month when using crossmatch results. Therefore, with the current practice, post-crossmatch matchmaking has a low information value, and matchmaking can be performed based on pre-crossmatch matchmaking, followed by a crossmatch.

TABLE 7
Performance of an implementation of one2seq neural
network 211 in the simulated environment not including
cross-match results, using MAE in months.

| Outcome/ | | | | |
|---|---|---|---|---|
| TimeHorizon | TH1 | TH2 | TH3 | TH4 |
| Death | 2.4 (2.10, | 6.5 (6.04, | 7.2 (6.80, | 8.1 (7.68, |
| | 2.70) | 6.96) | 7.60) | 8.52) |
| GF-AC | 1.9 (1.80, | 5.9 (5.68, | 6.8 (6.60, | 7.8 (7.54, |
| | 2.03) | 6.09) | 6.99) | 7.97) |
| GF-EOP | 1.3 (1.14, | 3.2 (2.85, | 4.0 (3.62, | 5.0 (4.57, |
| | 1.46) | 3.55) | 4.38) | 5.43) |
| GF-LOP | 3.0 (2.63, | 7.6 (7.12, | 8.3 (7.83, | 8.9 (8.42, |
| | 3.37) | 8.08) | 8.77) | 9.38) |
| GF-AR | 2.3 (2.07, | 6.3 (6.71, | 7.1 (6.77, | 7.4 (7.00, |
| | 2.53) | 5.89) | 7.49) | 7.80) |
| GF-CR | 3.4 (2.57, | 7.1 (6.61, | 7.7 (7.31, | 8.4 (7.97, |
| | 4.23) | 7.59) | 8.09) | 8.83) |
| GF-OC | 2.0 (1.71, | 6.5 (5.99, | 7.2 (6.74, | 9.2 (8.61, |
| | 2.29) | 7.01) | 7.66) | 9.79) |

Table 8 shows results of an implementation of seq2seq neural network 213 in a simulated environment using IMAE in months. For each IMAE in Table 8, a confidence interval at a 95% confidence level is also provided. The average error increases from about 5.3 months for the core performance (Table 6) to about 19.3 months (Table 8). The latter may be considered the real average performance of an implementation of seq2seq neural network 213 in real-life applications. It may be expected that an implementation of seq2seq neural network 213 has an average error of about 19.3 months in predicting the time of failure for patients who fail within 20 years after transplantation (TH4) when a part of data is given to an implementation of seq2seq neural network 213 (error is reduced by increasing the given data). The confidence interval is (18.59:20.01) for TH4, which means if the analysis is performed on new test sets, the IMAE for predictions may fall within the mentioned CI range, 95% of the time.

TABLE 8
Results of an implementation of seq2seq neural network 213 in a
simulated environment using IMAE in months. GF: Graft Failure

| Outcome/ | | | | |
|---|---|---|---|---|
| TimeHorizon | TH1 | TH2 | TH3 | TH4 |
| Death | 5.0 (4.20, | 11.3 (10.33, | 20.2 (18.9, | 24.0 (22.32, |
| | 5.80) | 12.27) | 21.5) | 25.68) |
| GF-AC | 4.5 (4.21, | 10.5 (10.14, | 16.9 (16.37, | 19.3 (18.58, |
| | 4.79) | 10.86) | 17.43) | 20.21) |
| GF-EOP | 4.6 (3.93, | 10.5 (9.36, | 8.9 (7.16, | 17.1 (14.58, |
| | 5.27) | 11.64) | 10.64) | 19.62) |
| GF-LOP | 4.1 (3.55, | 10.4 (9.67, | 15.9 (14.83, | 17.2 (15.76, |
| | 4.65) | 11.13) | 16.97) | 18.64) |
| GF-AR | 4.7 (4.19, | 9.4 (8.77, | 13.6 (12.66, | 19.3 (18.21, |
| | 5.21) | 10.03) | 14.54) | 20.39) |
| GF-CR | 3.7 (2.62, | 12.7 (11.78, | 20.2 (18.98, | 22.8 (21.21, |
| | 4.78) | 13.62) | 21.42) | 24.39) |
| GF-OC | 4.6 (3.76, | 11.0 (10.17, | 18.4 (17.20, | 22.4 (20.77, |
| | 5.44) | 11.83) | 19.60) | 24.03) |

Table 9 shows preliminary results of implementations of Bayesian neural networks, presented as a mean of expected values for the entire test dataset for each TH. For example, for patients in the test dataset of an implementation of DCD module 209 in TH1, MAE is bounded in the narrow interval of [53.23−0.24, 53.23+0.24], indicating a high confidence for about 53.23 hours as the MAE metric. Table 9 shows statistical performance of implementations of GRU-D neural network 205, seq2seq neural network 207, one2seq neural network 211, and seq2seq neural network 213 for the test dataset. Smaller intervals for predictions show higher confidence for representing the mean of MAE as a performance measure, and vice versa. Furthermore, since Bayesian neural networks can generate multiple PDFs for each prediction, each prediction may have its own confidence interval for the MAE measure, individually.

TABLE 9
Performance of an implementation of system 200 with Bayesian
neural networks, using MAE in months with their variances.

| Outcome/ | | | | |
|---|---|---|---|---|
| TimeHorizon | TH1 | TH2 | TH3 | TH4 |
| Death | 5.0 (4.20, | 11.3 (10.33, | 20.2 (18.9, | 24.0 (22.32, |
| | 5.80) | 12.27) | 21.5) | 25.68) |
| GF-AC | 4.5 (4.21, | 10.5 (10.14, | 16.9 (16.37, | 19.3 (18.59, |
| | 4.79) | 10.86) | 17.43) | 20.01) |
| GF-EOP | 4.6 (3.93, | 10.5 (9.36, | 8.9 (7.16, | 17.1 (14.58, |
| | 5.27) | 11.64) | 10.46) | 19.62) |
| GF-LOP | 4.1 (3.55, | 10.04 (9.67, | 15.9 (14.83, | 17.2 (15.7, |
| | 4.65) | 11.13) | 16.97) | 18.64) |
| GF-AR | 4.7 (4.19, | 9.4 (8.77, | 13.6 (12.66, | 19.3 (18.21, |
| | 5.21) | 10.13) | 14.54) | 20.39) |
| GF-CR | 3.7 (2.62, | 12.7 (11.78, | 20.2 (18.98, | 22.8 (21.21, |
| | 4.78) | 13.62) | 21.42) | 24.39) |
| GF-OC | 4.6 (3.76, | 11.0 (10.17, | 18.4 (17.20, | 22.4 (20.77, |
| | 5.44) | 11.83) | 19.60) | 24.03) |

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

What is claimed is:

1. A method for identifying a plurality of intended organ donors among a plurality of organ donor candidates, the method comprising:
obtaining a donor clinical dataset by acquiring each donor clinical data in the donor clinical dataset from a respective organ donor candidate of the plurality of organ donor candidates hospitalized in an intensive care unit (ICU);
obtaining a recipient clinical dataset by acquiring each recipient clinical data in the recipient clinical dataset from a respective recipient candidate of a plurality of recipient candidates;
predicting, utilizing one or more processors, one of an in-hospital death or survival of an intended organ donor candidate of the plurality of organ donor candidates based on intended donor clinical data in the donor clinical dataset, the intended donor clinical data acquired from the intended organ donor candidate;
estimating, utilizing the one or more processors, a time of death of the intended organ donor candidate responsive to the in-hospital death of the intended organ donor candidate being predicted;
obtaining a paired donor-recipient by pairing, utilizing the one or more processors, the intended organ donor candidate with an intended recipient of the plurality of recipient candidates for organ transplantation based on the intended donor clinical data and the recipient clinical dataset responsive to the time of death being in a predefined time period;
estimating, utilizing the one or more processors, a probability of organ transplant success for the paired donor-recipient based on the intended donor clinical data and intended recipient clinical data in the recipient clinical dataset, the intended recipient clinical data acquired from the intended recipient; and
pairing, utilizing the one or more processors, the intended recipient with the plurality of intended organ donors for organ transplantation based on the probability of organ transplant success.

2. The method of claim 1, wherein each of predicting the one of the in-hospital death or the survival of the intended organ donor candidate and estimating the time of death comprises:
generating a gated recurrent unit with trainable decays (GRU-D) output from the intended donor clinical data by applying the intended donor clinical data to a GRU-D layer associated with a GRU-D neural network;
generating a hidden state from the GRU-D output by applying the GRU-D output to a recurrent neural network (RNN) associated with the GRU-D neural network, the RNN comprising a plurality of RNN layers;
generating a latent variable from the hidden state, comprising:
generating a first (1^{st}) dense output of a plurality of dense outputs from the hidden state by applying the hidden state to a first (1^{st}) dense layer of a plurality of dense layers associated with the GRU-D neural network;
generating a first (1^{st}) dropout output of a plurality of dropout outputs by applying a dropout process on the 1^{st} dense output;
generating an n^{th} dense output of the plurality of dense outputs from an (n−1)^{th} dropout output of the plurality of dropout outputs by applying the (n−1)^{th} dropout output to an n^{th} dense layer of the plurality of dense layers where 1<n≤N_{d} and N_{d} is a number of the plurality of dense layers; and
generating an n^{th} dropout output of the plurality of dropout outputs from the n^{th} dense output by applying the dropout process on the n^{th} dense output, an N_{d}^{th} dropout output of the plurality of dropout outputs comprising the latent variable; and
generating one of a classification output comprising the one of the in-hospital death or the survival or a regression output comprising the time of death by applying an activation function to the latent variable.

3. The method of claim 2, wherein predicting the one of the in-hospital death or the survival of the intended organ donor candidate comprises training the GRU-D neural network by minimizing a loss function based on an ICU dataset, the loss function defined by the following:
$${L}_{classification} = {\sum }_{i = 1 , i ∈ {U}_{u}}^{{N}_{u}} {( {y}_{i , true}^{c} - {y}_{i , pred}^{c} )}^{2} / {N}_{u}$$
where:
L_{classification} is the loss function,
Uᵤ is a set of uncensored data in the ICU dataset,
Nᵤ is number of uncensored data in the set of uncensored data,
y_{i,true}^{c} is ground truth data for in-hospital death/survival classification of an i^{th} sample in the set of uncensored data, and
y_{i,pred}^{c} is a predicted value for in-hospital death/survival classification of the i^{th} sample.

4. The method of claim 3, wherein applying the activation function to the latent variable comprises applying a sigmoid function to the latent variable.

5. The method of claim 3, wherein training the GRU-D neural network comprises training a Bayesian neural network.

6. The method of claim 2, wherein estimating the time of death comprises training the GRU-D neural network by minimizing a loss function based on an ICU dataset, the loss function defined by the following:
$${L}_{regression} = {\sum }_{i = 1 , i ∈ {U}_{u}}^{{N}_{u}} {( {y}_{i , true}^{r} - {y}_{i , pred}^{r} )}^{2} / {N}_{u} + κ {\sum }_{i = j , j ∈ {U}_{c}}^{{N}_{c}} max ( 0 , {y}_{j , pred}^{r} - {y}_{j , c}^{r} ) / {N}_{c}$$
where:
L_{regression} is the loss function,
Uᵤ is a set of uncensored data in the ICU dataset,
Nᵤ is number of uncensored data in the set of uncensored data,
y_{i,true}^{r} is ground truth data for in-hospital time of death of an i^{th} uncensored sample in the set of uncensored data,
y_{i,pred}^{r} is a predicted value for in-hospital time of death of the i^{th} uncensored sample,
U_{c} is a set of censored data in the ICU dataset,
N_{c} is number of censored data in the set of censored data,
y_{j,pred}^{r} is a predicted value for in-hospital time of death of a j^{th} censored sample in the set of censored data,
y_{j,c}^{r} is a censoring time of the j^{th} censored sample, and
κ is a penalty coefficient.

7. The method of claim 6, wherein applying the activation function to the latent variable comprises applying a rectified linear unit (ReLU) function to the latent variable.

8. The method of claim 6, wherein training the GRU-D neural network comprises training a Bayesian neural network.

9. The method of claim 1, wherein estimating the time of death further comprises estimating a probability density function (PDF) of the time of death by:
generating a gated recurrent unit with trainable decays (GRU-D) output from the intended donor clinical data by applying the intended donor clinical data to a GRU-D layer associated with a sequence-to-sequence (seq2seq) neural network;
generating an encoded sequence from the GRU-D output by applying the GRU-D output to a first recurrent neural network (RNN) associated with the seq2seq neural network, the first RNN comprising a first plurality of RNN layers;
generating a decoded sequence associated with the time of death from the encoded sequence by applying the encoded sequence to a second RNN associated with the seq2seq neural network, the second RNN comprising a second plurality of RNN layers;
generating an event-related sequence associated with the time of death from the encoded sequence by applying an attention mechanism on the encoded sequence based on the decoded sequence;
generating a concatenated sequence by concatenating the event-related sequence and the decoded sequence; and
generating the PDF of the time of death from the concatenated sequence by applying the concatenated sequence to a time distributed dense layer associated with the seq2seq neural network.

10. The method of claim 9, wherein estimating the PDF of the time of death comprises training the seq2seq neural network by minimizing a forward loss function based on an ICU dataset, the forward loss function defined by the following:
$${L}_{forward} = {L}_{log} - ( {\sum }_{i = 1 , i ∈ {U}_{u}}^{{N}_{u}} {y}_{true}^{i} \times log {\sum }_{t = 1}^{{T}_{h}} {P}_{t}^{i} ) / {N}_{u}$$
where:
L_{forward} is the forward loss function,
L_{log} is a log-likelihood loss term,
Uᵤ is a set of uncensored data in the ICU dataset,
Nᵤ is number of uncensored data in the set of uncensored data, and
yₜᵣᵤₑⁱ is ground truth data for in-hospital time of death of an i^{th} uncensored sample in the set of uncensored data,
pₜⁱ is predicted likelihood for in-hospital time of death of the i^{th} uncensored sample at a time step t, and
Tₕ is a number of time steps in the PDF of the time of death.

11. The method of claim 10, wherein pairing the intended organ donor candidate with the intended recipient comprises:
training the seq2seq neural network by minimizing a reverse loss function based on the ICU dataset;
extracting a donor feature set from the intended donor clinical data utilizing the seq2seq neural network by applying the intended donor clinical data to the GRU-D layer;
extracting each of a plurality of recipient feature sets from a respective recipient clinical data in the recipient clinical dataset utilizing the seq2seq neural network by applying the respective recipient clinical data to the GRU-D layer;
grouping the donor feature set and a subset of the plurality of recipient feature sets in a donor cluster of a plurality of clusters by clustering the donor feature set and the plurality of recipient feature sets into the plurality of clusters based on distances between different feature sets among the donor feature set and the plurality of recipient feature sets;
obtaining a plurality of mean squared errors (MSEs) by calculating MSEs between the donor feature set and each of the plurality of recipient feature sets in the subset;
finding a smallest MSE among the plurality of MSEs, the smallest MSE associated with a most similar recipient feature set of the plurality of recipient feature sets in the subset to the donor feature set; and
pairing the intended organ donor candidate with a most similar recipient candidate of the plurality of recipient candidates to the intended organ donor candidate, the most similar recipient candidate associated with the most similar recipient feature set.

12. The method of claim 11, wherein minimizing the reverse loss function comprises minimizing a function defined by the following:
$${L}_{reverse} = {L}_{forward} + \frac{λ}{M} {\sum }_{m = 1}^{M} \vert {w}_{m} \vert$$
where:
Lᵣₑᵥₑᵣₛₑ is the reverse loss function,
λ is a regularization coefficient,

|wₘ| is an L₁ norm of a weight wₘ of an m^{th} training input of a plurality of training inputs in the ICU dataset, and
M is a number of the plurality of training inputs.

13. The method of claim 11, wherein each of training the seq2seq neural network by minimizing the forward loss function and training the seq2seq neural network by minimizing the reverse loss function comprises training a Bayesian neural network.

14. The method of claim 1, wherein estimating the probability of the organ transplant success for the paired donor-recipient comprises estimating a plurality of probability density functions (PDFs) for a plurality of events associated with the organ transplant success for the paired donor-recipient by:
generating a first (1^{st}) dense output of a plurality of dense outputs from the intended donor clinical data and the intended recipient clinical data by applying the intended donor clinical data and the intended recipient clinical data to a first (1^{st}) dense layer of a plurality of dense layers associated with a one-to-many (one2seq) neural network comprising a Bayesian neural network;
generating a first (1^{st}) dropout output of a plurality of dropout outputs by applying a dropout process to the 1^{st} dense output;
generating an m^{th} dense output of the plurality of dense outputs from an (m−1)^{th} dropout output of the plurality of dropout outputs by applying the (m−1)^{th} dropout output to an m^{th} dense layer of the plurality of dense layers where 1<m≤M_{d} and M_{d} is a number of the plurality of dense layers;
generating an m^{th} dropout output of the plurality of dropout outputs from the m^{th} dense output by applying the dropout process to the m^{th} dense output;
generating a normalized output by applying a batch normalization process to the an M_{d}^{th} dropout output of the plurality of dropout outputs;
generating a plurality of cause-specific outputs from the normalized output, the intended donor clinical data, and the intended recipient clinical data by applying the normalized output, the intended donor clinical data, and the intended recipient clinical data to a plurality of cause-specific subnetworks associated with the one2seq neural network, each of the plurality of cause-specific subnetworks comprising a respective plurality of gated recurrent unit (GRU) layers;
generating a concatenated sequence by concatenating the plurality of cause-specific outputs; and
generating each of the plurality of PDFs for each respective event of the plurality of events from the concatenated sequence by applying the concatenated sequence to a time distributed dense layer.

15. The method of claim 14, wherein estimating the plurality of PDFs comprises training the Bayesian neural network by minimizing a loss function defined by the following:
$${L}_{PDF} = {L}_{log} - ( {\sum }_{e = 1}^{{N}_{e}} {\sum }_{i = 1 , i ∈ {U}_{u}}^{{N}_{u}} {y}_{true}^{e , i} \times log ( {\sum }_{t = 1}^{{T}_{h}} {P}_{t}^{e , i} ) ) / {N}_{u}$$
where:
L_{PDF} is the loss function,
L_{log} is a log-likelihood loss term,
Nₑ is a number of the plurality of events,
Uᵤ is a set of uncensored data in the ICU dataset,
Nᵤ is number of uncensored data in the set of uncensored data,
yₜᵣᵤₑ^{e,i} is ground truth data of an i^{th} uncensored sample in the set of uncensored data for an event e of the plurality of events,
pₜ^{e,i} is predicted likelihood of the i^{th} uncensored sample for the event e at a time step t, and
Tₕ is a number of time steps in each of the plurality of PDFs.

16. The method of claim 14, wherein estimating the plurality of PDFs for the plurality of events comprises estimating each respective PDF of the plurality of PDFs for one of:
death time of the intended recipient;
a first graft failure due to early-onset pathologies (EOPs) of the intended recipient;
a second graft failure due to late-onset pathologies (LOPs) of the intended recipient;
a third graft failure due to acute rejection of the intended recipient's body;
a fourth graft failure due to chronic rejection of the intended recipient's body; and
a fifth graft failure due to other causes.

17. The method of claim 1, wherein pairing the intended recipient with the plurality of intended organ donors comprises:
training a sequence-to-sequence (seq2seq) neural network by minimizing a reverse loss function based on the ICU dataset;
extracting a recipient feature set from the intended recipient clinical data utilizing the seq2seq neural network by applying the intended recipient clinical data to the seq2seq neural network;
extracting each of a plurality of donor feature sets from a respective donor clinical data in the donor clinical dataset utilizing the seq2seq neural network by applying the respective donor clinical data to the seq2seq neural network;
grouping the recipient feature set and a subset of the plurality of donor feature sets in a recipient cluster of a plurality of clusters by clustering the recipient feature set and the plurality of donor feature sets into a plurality of clusters based on distances between different feature sets among the recipient feature set and the plurality of donor feature sets;
obtaining a plurality of mean squared errors (MSEs) by calculating MSEs between the recipient feature set and each of the plurality of donor feature sets in the subset;
extracting an MSE subset from the plurality of MSEs, each MSE in the MSE subset comprising a value smaller than an MSE threshold;
extracting an organ donor candidates subset from the plurality of organ donor candidates, each organ donor candidate in the organ donor candidates subset associated with a respective MSE in the MSE subset; and
pairing the intended recipient with each organ donor candidate in the organ donor candidates subset.

18. The method of claim 17, wherein each of extracting the recipient feature set by applying the intended recipient clinical data to the seq2seq neural network and extracting each of the plurality of donor feature sets by applying a respective donor clinical data to the seq2seq neural network comprises estimating a plurality of probability density functions (PDFs) for a plurality of events associated with one of the intended recipient or a respective organ donor candidate of the plurality of organ donor candidates from input data comprising one of the intended recipient clinical data or the respective donor clinical data, estimating the plurality of PDFs comprising:
generating a gated recurrent unit with trainable decays (GRU-D) output from the input data by applying the input data to a GRU-D layer associated with the seq2seq neural network;
generating an encoded sequence from the GRU-D output by applying the GRU-D output to an encoder recurrent neural network (RNN) associated with the seq2seq neural network, the encoder RNN comprising a first plurality of RNN layers;
generating a plurality of decoded sequences from the encoded sequence by applying the encoded sequence to a plurality of decoder RNNs associated with the seq2seq neural network, each of the plurality of decoder RNNs comprising a respective second plurality of RNN layers;
generating a plurality of event-related sequences from the encoded sequence by applying an attention mechanism to the encoded sequence based on a respective decoded sequence of the plurality of decoded sequences;
generating a plurality of concatenated sequences by concatenating each of the plurality of event-related sequences and a respective decoded sequence of the plurality of decoded sequences; and
generating each of the plurality of PDFs for each respective event of the plurality of events from a respective concatenated sequence of the plurality of concatenated sequences by applying each of the plurality of concatenated sequences to a respective time distributed dense layer.

19. The method of claim 18, wherein estimating the plurality of PDFs for the plurality of events comprises estimating each respective PDF of the plurality of PDFs by estimating one of:
death time;
a first graft failure due to early-onset pathologies (EOPs);
a second graft failure due to late-onset pathologies (LOPs);
a third graft failure due to acute rejection;
a fourth graft failure due to chronic rejection; and
a fifth graft failure due to other causes.

20. The method of claim 18, wherein training the seq2seq neural network comprises training a Bayesian neural network.
